(19) United States
(12) Patent Application Publication
(10) Pub. No.: US 2013/0303539 A1
(43) Pub. Date: Nov. 14, 2013

(54) COMPOUNDS WITH TRPV4 ACTIVITY, COMPOSITIONS AND ASSOCIATED METHODS THEREOF

(71) Applicants: David Krizaj, Salt Lake City, UT (US); Glenn D. Prestwich, Eastsound, WA (US); Peter Barabas, Salt Lake City, UT (US); Yong Xu, Midvale, UT (US); Daniel Ryskamp, Salt Lake City, UT (US)

(72) Inventors: David Krizaj, Salt Lake City, UT (US); Glenn D. Prestwich, Eastsound, WA (US); Peter Barabas, Salt Lake City, UT (US); Yong Xu, Midvale, UT (US); Daniel Ryskamp, Salt Lake City, UT (US)

(21) Appl. No.: 13/841,790

(22) Filed: Mar. 15, 2013

Related U.S. Application Data
(60) Provisional application No. 61/646,089, filed on May 11, 2012.
(60) Provisional application No. 61/714,691, filed on Oct. 16, 2012.

(51) Int. Cl. C07D 207/34; A61K 45/06; A61K 31/5377

(52) U.S. Cl. CPC C07D 207/34; A61K 31/5377; A61K 45/06
USPC 514/236.2; 544/141; 514/235.5; 544/143

(57) ABSTRACT

Compounds, compositions and methods useful for treating ocular diseases are provided. In particular, antagonists of TRPV4, their synthesis, pharmaceutical compositions thereof and methods of treating ocular diseases such as glaucoma, are disclosed.

### CROSS-REFERENCE TO RELATED APPLICATIONS

This application claims the benefit of U.S. Provisional Application Ser. Nos. 61/714,691 filed on Oct. 16, 2012 and 61/646,089 filed on May 11, 2012, both of which are herein incorporated by reference in their entirety.

### ACKNOWLEDGEMENT OF GOVERNMENT SUPPORT

This invention was made with U.S. government support from grants R01EY13870, EY022076, T32DC008553, and P30EY014800, as administered by the National Institutes of Health, and grant W81XWH-12-1-0244 as administered by the MEDCOM USAMRMC program at the U.S. Department of Defense. The U.S. government has certain rights in this invention.

### TECHNICAL FIELD

This disclosure relates to compounds, their synthesis, pharmaceutical compositions thereof and methods useful in the treatment of ocular diseases. More specifically, the disclosure relates to compounds, their synthesis, pharmaceutical compositions thereof, and methods comprising compounds with TRPV4 activity which are useful in the treatment of glaucoma and related diseases.

### BRIEF DESCRIPTION OF THE DRAWINGS

FIG. 1 shows that the TRPV4 is functional in cells of the human trabecular meshwork.

FIG. 2 shows the amount of RGC degeneration that occurs with prolonged blockage of fluid release.

FIG. 3 shows the amount of IOP that is blocked by injection of an exemplary TRPV4 antagonist.

FIG. 4 shows that IOP cannot be elevated in TRPV4 KO mice which lack the mechanosensitive mechanism.

FIG. 5 shows the average amount of IOP that occurs in the presence and absence of an exemplary TRPV4 antagonist.

FIG. 6 shows the relationship of RGC death and IOP in the presence and absence of an exemplary TRPV4 antagonist.

FIG. 7 highlights the location of affected TUJ-1 cells in relation to the optic nerve in the presence and absence of an exemplary TRPV4 antagonist.

FIG. 8 highlights the location of affected CFP+ cells in relation to the optic nerve in the presence and absence of an exemplary TRPV4 antagonist.

FIG. 9 shows the reduction of IOP in the presence of an exemplary TRPV4 antagonist.

FIG. 10 shows the reduction of IOP in the presence of multiple exemplary TRPV4 antagonists.

FIG. 11 shows the duration of IOP reduction in the presence of an exemplary TRPV4 antagonist.

FIG. 12 illustrates a comparison of the time course of exemplary TRPV4 antagonists.

FIG. 13 shows that a TRPV4 agonist evokes sustained increases in intracellular calcium concentration [Ca⁺⁺]ᵢ in retinal Müller glial cells and this effect is blocked by an exemplary TRPV4 antagonist.

FIG. 14 shows that steps of pressure from 10 mm Hg to 50 mm Hg induce inward currents from a retinal ganglion cell.

FIG. 15 shows the effect of an exemplary TRPV4 antagonist blocking the effect of a TRPV4 agonist.

FIG. 16 shows the effect of a TRPV4 agonist on cation influx into mouse retinal cells of the mouse retina.

FIG. 17 shows that cell swelling is accompanied by a pressure-dependent influx of calcium into retinal ganglion cells.

FIG. 18 shows the effect of hypotonic stimuli on cell swelling as measured by change in cell area.

FIG. 19 shows the effectiveness of an exemplary compound as a blocker of TRPV4 agonist-induced calcium responses.

FIG. 20 shows that Müller cells treated with 10 μM arachidonic acid respond with an increase in [Ca2+]ᵢ.

FIG. 21 shows that the tissues in the anterior chamber of the eye express the TRPV4 channel.

FIG. 22 shows that in vivo intraocular injection of a TRPV4 agonist substantially reduces the number of retinal ganglion cells.

FIG. 23 shows that an exemplary compound rescues cells from apoptosis.

FIG. 24 shows that the human retina shows similar TRPV4 expression compared to the mouse retina.

### DETAILED DESCRIPTION

The present disclosure provides compounds, compositions and methods of synthesizing antagonists of the transient receptor potential vanilloid 4 (TRPV4) ion channel that are effective against ocular disease, including glaucoma. The compounds act in a novel manner, in that not they not only decrease intraocular pressure (IOP), but also protect retinal ganglion nerve cells (RGCs) from cell death. Also disclosed are methods of using the described compositions to treat ocular disease.

The invention relates to the use of pharmaceutical compositions having one or more compounds of Formula I as the active ingredient, for treating ocular diseases.

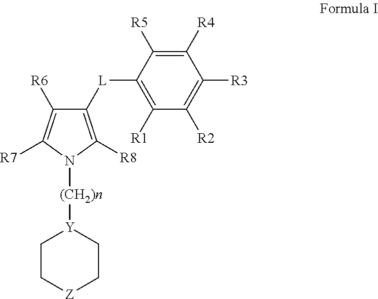

When the pharmaceutical composition is administered to a subject desiring or needing such treatment, it provides a reduction in, or lessening of an increase of, IOP associated with ocular diseases such as glaucoma, with a concomitant protection of RGCs from the cell death which normally results in such diseases. The compositions of the invention may be formulated with one or more pharmaceutically acceptable excipients, salts, or carriers. The pharmaceutical compositions of the invention may be delivered intraocularly, topically, or periocularly, for example, via an injection or gel-forming solution dosage form. The pharmaceutical compositions can be used in methods for treating and prophylaxis of ocular diseases such as glaucoma, other diseases characterized by abnormal IOP and/or RGC death, and indications for which an antagonist of TRPV4 may be indicated.

The invention therefore provides compounds of Formula I as described herein, methods of their synthesis, and pharmaceutical compositions comprising such compounds. In a specific use, the compounds can be used for the treatment and/or prophylaxis of ocular disease. Without being limited to any one theory, the inventors have found that compounds of Formula I as described herein have a dual action, as they both reduce IOP and protect RGCs from the cell death characteristic of ocular disease. The regulation of IOP by modulating fluid production in the anterior eye and neuronal cell loss in the posterior eye is a novel dual approach for treating ocular diseases, particularly glaucoma. Standard glaucoma therapies are designed to lower IOP in the anterior eye through eye drops that decrease the rate of fluid production. Neuroprotection in the posterior eye, however, is a critical unmet need.

In addition to the compounds of Formula I, the invention includes pharmaceutically acceptable prodrugs, pharmaceutically active metabolites, and pharmaceutically acceptable salts of such compounds.

Methods for treating an ocular condition associated with RGC death are included in the invention. Such methods can include delivering a TRPV4 antagonist into an eye of the subject such that the TRPV4 antagonist protects the RGCs from apoptotic cell death. Pharmaceutical compositions for treating an ocular condition associated with RGC death can include a TRPV4 antagonist dispersed in a pharmaceutically acceptable carrier, wherein the composition is formulated for ocular delivery.

Devices for treating an ocular condition associated with RGC death can include a housing having a structure configured to conform to an eye of a subject, and a TRPV4 antagonist dispersed in a pharmaceutical carrier contained in a reservoir located within the housing, wherein the reservoir is positioned to deliver the TRPV4 antagonist to the eye during use, are contemplated herein.

### I. DEFINITIONS

Unless specifically defined otherwise, the technical terms, as used herein, have their normal meaning as understood in the art. The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

As used herein, the singular terms “a,” “an,” and “the” include plural referents unless the context clearly indicates otherwise. Thus, for example, reference to “a channel” includes one or more of such channels. Similarly, the word “or” is intended to include “and” unless the context clearly indicates otherwise. Also, as used herein, the term “comprises” means “includes.” Hence “comprising A or B” means including A, B, or A and B.

“Administration of” and “administering a” compound refers to providing a compound, a prodrug of a compound, or a pharmaceutical composition comprising a compound as described herein. The compound or composition can be administered by another person to the subject or it can be self-administered by the subject.

As used herein, the term “agonist” refers to a compound that binds to a receptor or channel and triggers a response, and often mimics the action of the naturally occurring endogenous ligand. The potency of an agonist may be quantified by its EC₅₀ value.

As used herein, the term “antagonist” refers to a compound that binds to a receptor or channel but does not provoke a biological response, instead blocking or dampening an agonist-mediated response. Antagonists may act via binding at the receptor active site or at an allosteric site. In general, antagonists bind tightly to a receptor (i.e. have high affinity) but are devoid of activity (i.e. have no efficacy).

As used herein, the term “acetyl” group refers to a —C(═O)CH₃ group.

As used herein, the term “aldehyde” group refers to a carbonyl group where R″ is hydro.

As used herein, the term “alkyl” refers to a saturated aliphatic hydrocarbon including straight chain, cycloalkyl and branched chain groups. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc. up to and including 20 carbon atoms). The alkyl group may be a medium size alkyl group having 1 to 10 carbon atoms. It may be a lower alkyl group having 1 to 6 carbon atoms, or 1 to 4 carbon atoms. The alkyl group may be substituted or unsubstituted. When substituted, the substituent group(s) may be one or more individually selected from cycloalkyl, aryl, heteroaryl, heteroalicyclic, hydroxy, alkoxy, aryloxy, mercapto, alkylthio, arylthio, cyano, halo, carbonyl, carbamyl, amido, carboxy, nitro, silyl, and amino.

As used herein, the term “alkoxy” refers to both an —O-alkyl and an —O-cycloalkyl group, as defined herein. Lower alkoxy refers to —O-lower alkyl groups.

As used herein, the term “amino” refers to an —NRR′ group, with R and R′ both being hydro.

As used herein, the term “amido” refers to a —C(═O)NRR′ group with R and R′ being selected from the group consisting of hydro, alkyl, haloalkyl, aryl, heteroaryl and heterocycle as defined herein.

As used herein, the term “aryl” refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituted group(s) may be one or more selected from halo, haloalkyl, alkyl, hydroxy, alkoxy, haloalkoxy, mercapto, cyano, nitro, carbonyl, carboxyl, carbamyl, amido, sulfinyl, sulfonyl, and amino.

As used herein, the term “carbonyl” group refers to a —C(═O)R″ group, where R″ is selected from the group consisting of hydro, alkyl, haloalkyl, aryl, heteroaryl and heterocycle, as defined herein.

As used herein, the term “carboxyalkyl” group refers to an -alkyl-C(═O)OR″ group with R″ as defined above.

As used herein, the term “carboxyl” group refers to a —C(═O)OR″ group with R″ as defined above.

As used herein, the term “carboxyl salt” refers to a —C(═O)O⁻ M⁺ group wherein M⁺ is a metal ion. In certain embodiments, M⁺ is selected from the group consisting of lithium, sodium, magnesium, calcium, potassium, barium, iron, aluminum, zinc and quaternary ammonium.

As used herein, the term “carboxylic acid” refers to a carboxyl group in which R″ is hydro.

As used herein, the term “cyano” refers to a —C≡N group.

As used herein, the term “cycloalkyl” refers to an all-carbon monocyclic or fused ring (i.e., rings which share an adjacent pair of carbon atoms) group wherein one or more of the rings does not have a completely conjugated pi-electron system. Examples, without limitation, of cycloalkyl groups are cyclopropane, cyclobutane, cyclopentane, cyclopentene, cyclohexane, adamantane, cyclohexadiene, cycloheptane and cycloheptatriene. A cycloalkyl group may be substituted or unsubstituted. When substituted, the substituent group(s) may be one or more individually selected from alkyl, aryl, heteroaryl, heterocyclic, hydroxy, alkoxy, aryloxy, mercapto, cyano, halo, carbonyl, carboxyl, carbamyl, amido, nitro, and amino.

As used herein, the term “ester” is a carboxyl group, as defined above, wherein R″ is any of the listed groups other than hydro.

As used herein, the term “halo” refers to chloro, fluoro, bromo, and iodo.

As used herein, the terms “halogenated alkyl” or “haloalkyl group” refer to an alkyl group as defined above with one or more hydrogen atoms present on these groups substituted with a halogen (F, Cl, Br, I). In an embodiment, the haloalkyl group is —CF₃. A “haloalkoxy” group refers to a group with one or more hydrogen atoms present on an ether, such as a methyl ether (—OCH₃), substituted with one or more halogens. For example, a trifluoromethyl ether has a formula of —OCF₃.

As used herein, the term “heteroaryl” refers to groups having 5 to 14 ring atoms; 6, 10 or 14 pi electrons shared in a cyclic array; and containing carbon atoms and 1, 2 or 3 oxygen, nitrogen, phosphorus or sulfur heteroatoms. Non-limiting heteroaryl groups include thienyl (thiophenyl), benzo[b]thienyl, furyl (furanyl), isobenzofuranyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, phthalzinyl, naphthyridinyl, quinozalinyl, cinnolinyl, pteridinyl, carbazolyl, beta-carbolinyl, and isoxazolyl. Where the heteroaryl group contains a nitrogen atom in a ring, such nitrogen atom may be in the form of an N-oxide, e.g., a pyridyl N oxide, pyrazinyl N-oxide and pyrimidinyl N-oxide. When substituted, the substituted group(s) may be one or more selected from alkyl, cycloalkyl, halo, trihalomethyl, hydroxy, alkoxy, aryloxy, mercapto, alkylthio, arylthio, cyano, nitro, carbonyl, thiocarbonyl, sulfonamido, carboxy, sulfinyl, sulfonyl, carbamyl, amido, and amino.

As used herein, the term “heterocycle” or heterocyclic” refers to a saturated or partially saturated 3-7 membered monocyclic, or 7-10 membered bicyclic ring system, which consists of carbon atoms and from one to four heteroatoms independently selected from the group consisting of O, N, P, and S, wherein the nitrogen, phosphorus and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on carbon or on a nitrogen atom if the resulting compound is stable. Non-limiting saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, pyrazolidinyl, and pyrazolinyl groups. Example of “heterocycles” or “heterocyclic” rings also include, but are not limited to, morpholino, piperidyl, piperazinyl, pyrrolidinyl, thiomorpholino, homopiperazinyl, imidazolyl, imidazolidinyl, pyrazolidinyl, dioxanyl and dioxolanyl. “Heterocycle” can include heteroaryls when the pi-electron system of a heterocycle is completely conjugated.

Exemplary substituents of a heterocycle include halogen, cyano, nitro, oxo, amino, alkyl, alkoxy, haloalkyl, haloalkoxy, carboxyl, CO-alkyl, benzyloxy and pyrazolyl.

As used herein, the term “hydro” refers to a hydrogen atom (—H group).

As used herein, the term “hydroxy” refers to an —OH group.

As used herein, the term “mercapto” group refers to an —SH group.

As used herein, the term “nitro” refers to a —NO₂ group.

Optionally substituted groups, such as “optionally substituted alkyl,” refers to groups, such as an alkyl group, that when substituted, have 1, 2, 3, 4 or 5 substituents, typically 1, 2 or 3 substituents, selected from alkoxy, optionally substituted alkoxy, acyl, acylamino, acyloxy, amino, aminoacyl, aminoacyloxy, aryl, carboxylalkyl, optionally substituted cycloalkyl, optionally substituted cycloalkenyl, halogen, optionally substituted heteroaryl, optionally substituted heterocyclyl, hydroxy, sulfonyl, thiol and thioalkoxy. Optionally substituted alkyl groups include haloalkyl groups, such as fluoroalkyl groups, including, without limitation, trifluoromethyl groups and trifluoromethyl ethers.

As used herein, the term “quaternary ammonium” refers to a —⁺NR R′ R′″ group wherein R, R′, and R′″ are independently selected from the group consisting of hydro and unsubstituted alkyl.

“Saturated or unsaturated” includes substituents saturated with hydrogens, substituents completely unsaturated with hydrogens and substituents partially saturated with hydrogens.

As used herein, the term “sulfinyl” refers to a —S(═O)R″ group, wherein R″ is selected from the group consisting of hydro, alkyl, haloalkyl, aryl, heteroaryl and heterocycle, as defined herein.

As used herein, the term “sulfonyl” refers to a —S(═O)₂R″ group, with R″ as defined above.

As used herein, the term “sulfonamido” refers to a —S(═O)₂ NRR, with R″ and R′ being independently selected from the group consisting of hydro, alkyl, haloalkyl, aryl, heteroaryl and heterocycle, as defined herein.

As used herein, the term “thiocarbonyl” refers to a —C(═S)R″ group, with R″ as defined above.

“Coadminister” means that each of at least two compounds are administered during a time frame wherein the respective periods of biological activity overlap. Thus, the term includes sequential as well as coextensive administration of two or more drug compounds.

“Derivative” refers to a compound or portion of a compound that is derived from or is theoretically derivable from a parent compound.

As used herein, the term “dose” or “dosage” refers to the amount of active ingredient that a subject takes or is administered at one time. For example, a 100 mg dose of a compound of Formula I refers to, in the case of a twice-daily dosage regimen, a situation where the individual is administered 100 mg of a compound of Formula I twice a day, e.g., 100 mg in the morning and 100 mg in the evening. The 100 mg of a compound of Formula I dose can be divided into two or more dosage units, e.g., two 50 mg dosage units of a compound of Formula I as an injection or two 50 mg dosage units of a compound of Formula I as an eyedrop.

“Inhibiting” (which is inclusive of “treating”) refers to inhibiting the development of an ocular disease, for example, in a subject who is at risk for a disease such as glaucoma. “Inhibiting” also refers to any quantitative or qualitative reduction, including moderation of IOP or neuronal cell death, of

a measurable property relative to a control.

As used herein, the term “ocular disease” refers to any disease or disorder of the eye or pertaining to the eye, including infections thereof. For example, ocular diseases include various retinopathies including diabetic retinopathy, glaucoma, macular degeneration, and retinitis pigmentosa. The disease may be chronic, or it may be acute.

“Optional” or “optionally” means that the subsequently described event or circumstance can but need not occur, and that the description includes instances where said event or circumstance occurs and instances where it does not.

“A pharmaceutically acceptable prodrug” is a compound that may be converted under physiological conditions or by solvolysis to the specified compound or to a pharmaceutically acceptable salt of such compound.

A prodrug is an active or inactive compound that is modified chemically through an in vivo physiological action, such as hydrolysis or metabolism, into an active compound following administration of the prodrug to a subject. The suitability and techniques involved in making and using prodrugs are well known by those skilled in the art. The term “prodrug” also is intended to include any covalently bonded carriers that release an active parent drug of the present invention in vivo when the prodrug is administered to a subject. The compounds and compositions disclosed herein may be delivered in prodrug form. Thus, also contemplated are prodrugs of the presently disclosed compounds, methods of delivering prodrugs and compositions containing such prodrugs.

Prodrugs of the disclosed compounds may be prepared by modifying one or more functional groups present in the compound in such a way that the modifications are cleaved, either in routine manipulation or in vivo, to yield the parent compound. Prodrugs include compounds having a carboxyl group functionalized with any group that is cleaved in vivo to yield the corresponding carboxylic acid group. For a general discussion of prodrugs involving esters see Svensson and Tunek, Drug Metabolism Reviews 165 (1988), and Bundgaard, Design of Prodrugs, Elsevier (1985). Prodrugs and active metabolites of compound may be identified using routine techniques known in the art. See, e.g., Bertolini, G et al., J. Med. Chem., 40, 2011-2016 (1997); Shan, D. et al., J. Pharm. Sci., 86 (7), 756-767; Bagshawe K., Drug Dev. Res., 34, 220-230 (1995); Bodor N.; Advance in Drug Res., 13, 224-331 (1984); Larsen, I. K., Design and Application of Prodrugs, Drug Design and Development (Krogsgaard-Larsen et al., eds., Harwood Academic Publishers, 1991).

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

“A pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrophosphates, dihydrophosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, salicylates, suberates, sebacates, fumarates, maleates, butyne-1,4 dioates, hexyne-1,6-dioates, benzoates, chlorobenzoates, methyl benzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, and mandelates.

“Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide.

These salts may be prepared by standard procedures, for example by reaction of the free acid with a suitable organic or inorganic base. Any chemical compound recited in this specification may alternatively be administered as a pharmaceutically acceptable salt thereof.

“Pharmaceutically acceptable salts” are also inclusive of the free acid, base, and zwitterionic forms. Descriptions of exemplary pharmaceutically acceptable salts can be found in Stahl and Wermuth, Eds., Handbook of Pharmaceutical Salts; Properties, Selection and Use, Wiley VCH (2008). When compounds disclosed herein include an acidic function such as a carboxy group, then suitable pharmaceutically acceptable cation pairs for the carboxy group are well known to those skilled in the art and include alkaline, alkaline earth, ammonium, and quaternary ammonium cations. Such salts are known to those of skill in the art. For additional examples of “pharmacologically acceptable salts,” see Berge et al., J. Pharm. Sci. 66:1 (1977).

As used herein, the term “preventing an increase in a symptom” refers to both not allowing a symptom to increase or worsen, as well as reducing the rate of increase in the symptom. For example, a symptom can be an increase in intraocular pressure. Preventing an increase, according to the definition provided herein, means that the amount of symptom (e.g., pressure) does not increase or that the rate at which it increases is reduced.

As used herein, the term “preventing an ocular disease” refers to a slowing of the disease or of the onset of the disease or the symptoms thereof. Preventing an ocular disease can include stopping the onset of the disease or symptoms thereof.

As used herein, the term “treating an ocular disease” refers to a slowing of or a reversal of the progression of the disease. Treating an ocular disease includes treating a symptom and/or reducing the symptoms of the disease.

“Treatment” refers to a therapeutic intervention that ameliorates a sign or symptom of a disease or pathological condition after it has begun to develop. As used herein, the term “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease.

A “prophylactic” treatment is a treatment administered to a subject who does not exhibit signs of a disease or exhibits only early signs, for the purpose of decreasing the risk of developing pathology.

The term “subject” includes both human and veterinary subjects.

A “therapeutically effective amount” or refers to a quantity of a specified agent sufficient to achieve a desired effect in a subject being treated with that agent. For example, this may be the amount of a compound disclosed herein useful in treating glaucoma in a subject. A therapeutically effective amount of an agent is an amount sufficient to inhibit or treat the disease without causing substantial toxicity in the subject. The therapeutically effective amount of an agent will be dependent on the subject being treated, the severity of the affliction, and the manner of administration of the therapeutic composition. Methods of determining a therapeutically effective amount of the disclosed compound sufficient to achieve a desired effect in a subject with glaucoma will be understood by those of skill in the art in light of this disclosure.

As used herein, the term “unit dosage form” refers to a physically discrete unit, such as a preloaded injector, suitable as a unitary dosage for a human patient. Each unit contains a predetermined quantity of a compound of Formula I, which was discovered or believed to produce the desired pharmacokinetic profile which yields the desired therapeutic effect. The dosage unit is composed of a compound of Formula I in association with at least one pharmaceutically acceptable carrier, salt, excipient, or combination thereof.

References cited throughout this disclosure, including journal articles and patents, are herein incorporated by reference.

It is noted that some forms of glaucoma do not appear to be associated with an increase in IOP, but such “normal-tension” glaucomas appear to generate similar ocular degeneration as traditional glaucoma. As such, reference to glaucoma herein additionally refers to normal-tension glaucoma unless the context clearly indicates otherwise. It is also noted that the present scope is not limited to glaucoma or conditions causing or resulting from an increase in IOP, but rather also includes conditions associated with RGC apoptosis, ocular degeneration, and ocular trauma.

### II. COMPOUNDS AND METHODS OF SYNTHESIS

In general, the invention relates to compounds of Formula I, pharmaceutically acceptable salts thereof, and pharmaceutical compositions containing the same. The compounds of the invention can be used for the treatment and prophylaxis of ocular diseases, including glaucoma.

It is noted that the scope of the present disclosure, however, includes any TRPV4 antagonist capable of treating TRPV4-related ocular conditions.

In an embodiment, the compounds disclosed herein include compounds having the structure shown below in Formula I:

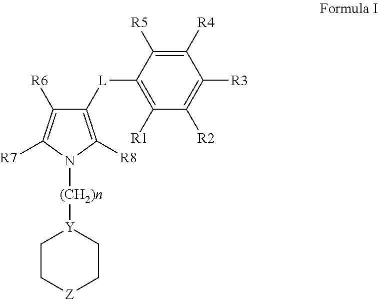

In the compounds of Formula I, the group labeled L may be C(═O)NR10, SO₂NR10, a C1-C6 alkylene, or a bond. The group labeled Y may be N or CR10. The group labeled Z may be O, NR10, S, SO₂ or C(R10)₂. The group labeled n may be 0, 1, 2, 3, 4, 5, or 6. In some embodiments, L is C(═O)NHR10 or SO₂NR10, Y is N, and Z is O, N(C1-C6 alkyl), SO₂ or CH₂. In certain embodiments, L is C(═O)NH, Y is N and Z is O. In further embodiments, n is 3. In an embodiment, Y is N which has been quaternized as a N-phosphooxymethyl prodrug; R3—N⁺—CH₂—OPO(OH)₂.

The groups labeled R1, R2, R3, R4, and R5 may be independently selected from at least one of hydro, alkyl, haloalkyl, hydroxy, alkoxy, haloalkoxy, cyano, carboxyl, or amido. In some embodiments, R1, R3, R4 and R5 are hydro and R2 is haloalkyl, haloalkoxy, carboxyl, cyano or amido. For example, R1, R3, R4 and R5 are hydro and R2 is OCF₃, CF₃, CONR₂ or COOR, where R is hydro or alkyl. In certain embodiments, R1, R3, R4 and R5 are hydro and R2 is an isopropyl or ethyl ester. In some embodiments, R is lower alkyl, C1-C6 alkyl, C1-C4 alkyl, methyl, ethyl or isopropyl.

The group labeled R6 may be hydro, alkyl, haloalkyl, heterocyclic or aryl, or may be connected to R7 via a cyclic ring system fused with the pyrrole ring shown, to form a bicyclic core. The additional ring may be substituted or unsubstituted, and may be aromatic or heteroaromatic. In an embodiment, R6 and R7 are connected to form an indole core.

The group labeled R7 may be hydro, alkyl, haloalkyl, heterocyclic or aryl, or may be connected to R6 via a cyclic ring system fused with the pyrrole ring shown, to form a bicyclic core. The additional ring may be substituted or unsubstituted, and may be aromatic or heteroaromatic. In an embodiment, R7 and R6 are connected to form an indole core. In certain embodiments, R7 is phenyl.

The group labeled R8 may be hydro, alkyl, haloalkyl, heterocyclic or aryl. R8 may be substituted or unsubstituted. In an embodiment, R8 is alkyl. In certain embodiments, R8 is lower alkyl, C1-C6 alkyl, C1-C4 alkyl, or methyl. In an embodiment, R8 is methyl.

The group labeled as R10 may be hydro, alkyl, haloalkyl, carboxyalkyl, carboxyl, alkyl methylene carbonate, methylene carbamyl, thiophenyl or —S-carboxyalkyl. In certain embodiments, R10 is hydro. In some embodiments, R10 is lower alkyl, C1-C6 alkyl, C1-C4 alkyl, methyl, ethyl or isopropyl.

In certain embodiments, the invention relates to compounds of Formula I wherein L is C(═O)NR10, SO₂NR10, a C1-C6 alkylene, or a bond; Y is N or CR10; Z is O, NR10, S, SO₂ or C(R10)₂; n is 0, 1, 2, 3, 4, 5, or 6; R1, R2, R3, R4, and R5 are independently selected from at least one of hydro, alkyl, haloalkyl, hydroxy, alkoxy, haloalkoxy, cyano, carboxyl, or amido; R6 and R7 are independently selected from at least one of hydro, alkyl, haloalkyl, heterocyclic or aryl, or R6 and R7 are connected via a cyclic ring system; R8 is hydro, alkyl, haloalkyl, heterocyclic or aryl; and R10 is hydro, alkyl, or haloalkyl.

In some embodiments, the invention relates to compounds of Formula I wherein L is C(═O)NH or SO₂NH; Y is N; Z is O, N(C1-C6 alkyl), SO₂ or CH₂; n is 3; R1, R3, R4 and R5 are hydro; R2 is haloalkyl, haloalkoxy, carboxyl, cyano or amido; R6 is hydro, R7 is aryl, or R6 and R7 are connected via a cyclic ring system; and R8 is alkyl.

In an embodiment, the invention relates to compounds of Formula I wherein L is C(═O)NH; Y is N; Z is O; n is 3; R1, R3, R4 and R5 are hydro; R2 is OCF3, CF3, CONR2 or COOR, where R is hydro or alkyl; R6 is hydro, R7 is phenyl, or R6 and R7 are connected via a cyclic ring system; and R8 is methyl.

In an embodiment, exemplary compounds include compounds having the structure shown below in Formula II, containing a pyrrole core structure:

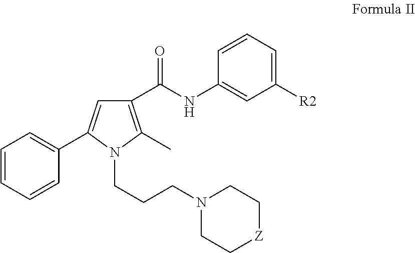

In the compounds of Formula II, the group labeled Z may be O, NR10, S, SO₂ or C(R10)₂. In some embodiments, Z is O, N(C1-C6 alkyl), SO₂ or CH₂. In certain embodiments, Z is O.

The group labeled R2 may be selected from at least one of hydro, alkyl, haloalkyl, hydroxy, alkoxy, haloalkoxy, cyano, carboxyl, or amido. In some embodiments, R2 is haloalkyl, haloalkoxy, carboxyl, cyano or amido. For example, R2 is OCF₃, CF₃, CONR₂ or COOR, where R is hydro or alkyl. In certain embodiments, R2 is an isopropyl or ethyl ester.

R10 may be hydro, alkyl, or haloalkyl. In certain embodiments, R10 is hydro. In some embodiments, R10 is lower alkyl, C1-C6 alkyl, C1-C4 alkyl, methyl, ethyl or isopropyl.

In certain embodiments, the invention relates to compounds of Formula II wherein Z is O, NR10, S, SO₂ or C(R10)₂; R2 is selected from at least one of hydro, alkyl, haloalkyl, hydroxy, alkoxy, haloalkoxy, cyano, carboxyl, or amido; and R10 is hydro, alkyl, or haloalkyl.

In some embodiments, the invention relates to compounds of Formula II wherein Z is O, N(C1-C6 alkyl), SO₂ or CH₂; and R2 is OCF₃, CF₃ or COOR, wherein R is hydro or alkyl.

In an embodiment, the invention relates to compounds of Formula II wherein Z is O; and R2 is OCF₃, CF₃, or COOR, wherein R is isopropyl.

In an embodiment, exemplary compounds also include compounds having the structure shown below in Formula III, containing an indole core structure:

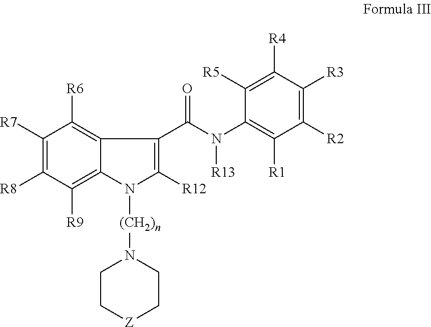

In the compounds of Formula III, the group labeled Z may be O, NR10, S, SO₂ or C(R10)₂. In some embodiments, Z is O, N(C1-C6 alkyl), SO₂ or CH₂. In certain embodiments, Z is O. The group labeled n may be 0, 1, 2, 3, 4, 5, or 6. In some embodiments, n is 3.

The groups labeled R1-R9 and R12 may be selected from at least one of hydro, alkyl, haloalkyl, hydroxy, alkoxy, haloalkoxy, cyano, carboxyl, or amido. In some embodiments, R2 is haloalkyl, haloalkoxy, carboxyl, cyano or amido. For example, R2 is OCF₃, CF₃, CONR₂ or COOR, where R is hydro or alkyl. In certain embodiments, R2 is an isopropyl or ethyl ester. In an embodiment, R8 is halo or alkyl. In some embodiments, R8 is lower alkyl, C1-C6 alkyl, or C1-C4 alkyl. In further embodiments, R8 is chloro or tert-butyl.

The group labeled R10 may be hydro, alkyl, or haloalkyl. In certain embodiments, R10 is hydro. In some embodiments, R10 is lower alkyl, C1-C6 alkyl, C1-C4 alkyl, methyl, ethyl or isopropyl.

The group labeled R13 may be hydro, alkyl, haloalkyl, carboxyalkyl, carboxyl, alkyl methylene carbonate, methylene carbamyl, thiophenyl or —S-carboxyalkyl.

In certain embodiments, the invention relates to compounds of Formula III wherein Z is O, NR10, S, SO₂ or C(R10)₂; R2 is selected from at least one of hydro, alkyl, haloalkyl, hydroxy, alkoxy, haloalkoxy, cyano, carboxyl, or amido; R8 is hydro, alkyl, or haloalkyl; R10 is hydro, alkyl, or haloalkyl; R12 is alkyl; and R13 is hydro.

In some embodiments, the invention relates to compounds of Formula III wherein Z is O, N(C1-C6 alkyl), SO₂ or CH₂; R2 is OCF₃, CF₃ or COOR, wherein R is hydro or alkyl; R8 is halo or alkyl; R12 is methyl; and R13 is hydro.

In an embodiment, the invention relates to compounds of Formula III wherein Z is O; R2 is OCF₃, CF₃, or COOR, wherein R is isopropyl, ethyl, butyl, isobutyl, n-propyl or methyl; R8 is chloro or tert-butyl; R12 is methyl; and R13 is hydro.

In certain embodiments, the compound of Formula I is Compound 1 or an HCl salt thereof:

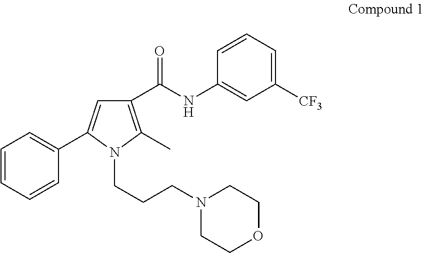 [SMILES: Cc1c(C(=O)Nc2cccc(C(F)(F)F)c2)cc(-c2ccccc2)n1CCCN1CCOCC1]

In further embodiments, the compound of Formula I is Compound 2 or Compound 3, or HCl salts thereof:

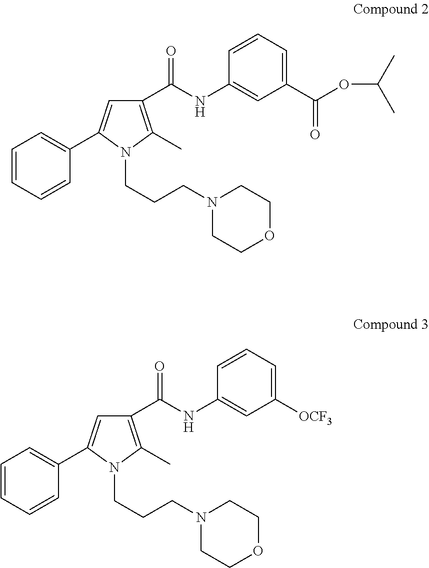 [SMILES: Cc1c(C(=O)Nc2cccc(C(=O)OC(C)C)c2)cc(-c2ccccc2)n1CCCN1CCOCC1 ; Cc1c(C(=O)Nc2cccc(OC(F)(F)F)c2)cc(-c2ccccc2)n1CCCN1CCOCC1]

In an embodiment, the compound of Formula I is Compound 5, or HCl salts thereof:

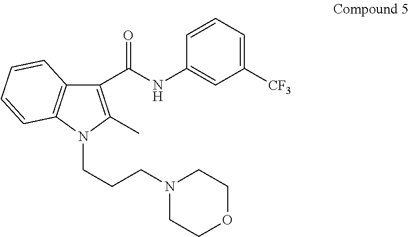 [SMILES: Cc1c(C(=O)Nc2cccc(C(F)(F)F)c2)c2ccccc2n1CCCN1CCOCC1]

Some of the compounds of Formula I for use in the invention may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Accordingly, compounds and compositions may be provided as individual pure enantiomers or as stereoisomeric mixtures, including racemic mixtures. In certain embodiments, the compounds disclosed herein may be synthesized in or are purified to be in a substantially enantiopure form, such as in a 90% enantiomeric excess, a 95% enantiomeric excess, a 97% enantiomeric excess or even in greater than a 99% enantiomeric excess, such as in enantiopure form. Furthermore, some of the compounds may exist as cis and trans geometric isomers; all such isomers and mixtures thereof are intended to be within the scope of the present invention.

The compounds of the present invention can be synthesized using the methods as described herein, together with synthetic methods known in the art of synthetic organic chemistry or variations thereon as appreciated by those skilled in the art. It will be appreciated that where typical process conditions (i.e., reaction temperatures, times, molar ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C NMR), infrared spectroscopy, spectrophotometry (e.g., UV-visible), mass spectrometry, or by chromatography such as high performance liquid chromatography (HPLC) or thin layer chromatography (TLC).

Additionally, the formulas are intended to cover solvated as well as unsolvated forms of the identified structures. For example, the compounds of Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

Representative synthetic schemes and experimental descriptions for the compounds of Formula I for use in the methods of the invention are provided below.

An exemplary synthetic pathway for Compound 1 is outlined in Scheme 1. Reaction of ethyl acetoacetate 2 with sodium hydride in THF, followed by the addition of 2-bromo-1-phenylethanone 1 gave the 1,4-diketone 3 in nearly qualitative yield. The pyrrole cyclization step was performed following a Paal-Knorr cyclization between the 1,4-diketone 3 and ammonium acetate in acetic acid at 80° C., to afford the pyrrole ethyl ester 4. Alkylation of the pyrrole nitrogen was achieved by the reaction of pyrrole ethyl ester 4 with 4-(3-chloropropyl)morpholine 5 in the presence of NaH. The yield for this step was low and much of the pyrrole starting material was recovered. Hydrolysis of the ethyl ester 6 with potassium hydroxide in refluxing EtOH yielded the corresponding carboxylic acid 7. The amidation reaction with amine 8 proceeded well under activated ester conditions (EDCl/HOAt) to provide Compound 1.

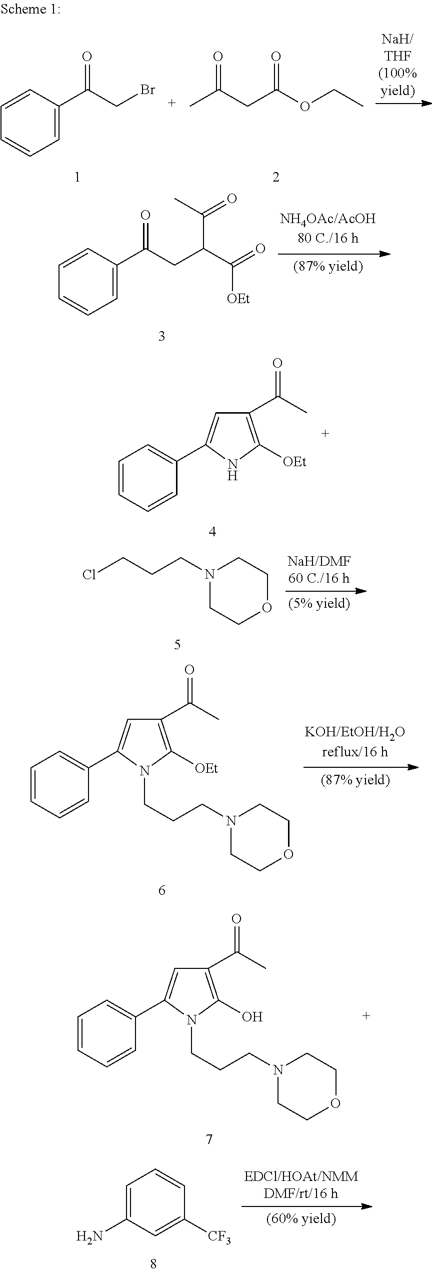

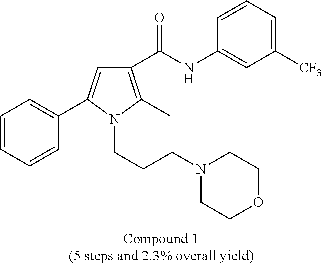 [SMILES: Cc1c(C(=O)Nc2cccc(C(F)(F)F)c2)cc(-c2ccccc2)n1CCCN1CCOCC1]

Intermediate 3; ethyl 2-acetyl-4-oxo-4-phenyl butanoate.

To a solution of ethyl acetoacetate (1.8 g; 13.8 mmol) in 15 mL anhydrous THF was added NaH (0.33 g; 13.8 mmol) at room temperature (rt) under nitrogen. The reaction mixture was stirred for 30 min, and 2-bromoacetophenone (2.5 g; 12.6 mmol) was added dropwise. After stirring at rt for 4 h, the reaction was quenched with water (20 mL). The reaction mixture was extracted with ethyl acetate (30 mL×2). The combined organic phases were washed with saturated aqueous ammonium chloride (20 mL), and dried over anhydrous sodium sulfate. After removal of the solvent, the product 3 was dried under vacuum and used for the next step without further purification (3.12 g; 13.8 mmol, 100% yield). ¹H-NMR (CDCl₃/400 MHz): δ 7.95 (d, 2H); 7.54 (m, 1H); 7.45 (m, 2H); 4.21 (m, 3H); 3.70 (m, 1H); 3.52 (m, 1H); 2.43 (s, 3H); 1.26 (m, 3H).

Intermediate 4; ethyl 2-methyl-5-phenyl-1H-pyrrole-3-carboxylate.

Ethyl 2-acetyl-4-oxo-4-phenylbutanoate 3 (3.1 g; 12.6 mmol) was treated with NH₄OAc (4.8 g; 63.0 mmol) in acetic acid (15 mL) at room temperature. After stirring for 10 min, the reaction mixture was heated to 80° C. overnight. After cooling to rt, the acetic acid was evaporated under reduced pressure, and water (20 mL) was added. The reaction mixture was extracted with ethyl acetate. Purification of the residue by silica gel chromatography provided product 4 (3.5 g; 10.92 mmol, 87% yield). ¹H-NMR (CDCl₃/400 MHz): δ 8.70 (br, 1H); 7.43 (m, 2H); 7.35 (m, 2H); 7.20 (m, 1H); 6.82 (s, 1H); 4.23 (m, 2H); 2.48 (s, 3H); 2.05 (m, 2H); 1.33 (m, 3H).

Intermediate 6; ethyl 2-methyl-1-(3-morpholinopropyl)-5-phenyl-1H-pyrrole-3-carboxylate.

A solution of ethyl 2-methyl-5-phenyl-1H-pyrrole-3-carboxylate 4 (200 mg; 0.873 mmol) in DMF (2 mL) was treated with NaH in mineral oil (100 mg; 4.64 mmol) and the mixture was stirred at rt for 1 hour. Following the addition of 4-(3-chloropropyl)morpholine 5, the mixture was stirred at 50° C. for 4 hours. After cooling to rt, the reaction mixture was quenched with water (20 mL). The reaction mixture was extracted with ethyl acetate. Purification by silica gel chromatography provided product 6 (15 mg; 0.044 mmol, 5% yield). ¹H-NMR (CD₃OD/400 MHz): δ 7.42 (m, 2H); 7.35 (m, 3H); 6.42 (s, 1H); 4.23 (m, 2H); 4.04 (m, 2H); 3.53 (m, 4H); 2.60 (s, 3H); 2.14 (m, 6H); 1.61 (m, 2H); 1.33 (m, 3H). MS (ES⁺, m/z): 357.3 (M⁺+1, 100.0).

Intermediate 7; 2-methyl-1-(3-morpholinopropyl)-5-phenyl-1H-pyrrole-3-carboxylic acid.

To a solution of ethyl 2-methyl-1-(3-morpholinopropyl)-5-phenyl-1H-pyrrole-3-carboxylate 6 (1.0 g; 2.81 mmol) in a mixture of EtOH (5 mL) and water (1 mL), was added KOH (1.57 g; 28.1 mmol) at rt. The reaction mixture was refluxed overnight. The EtOH was evaporated under reduced pressure. Water (10 mL) was added and the solution was neutralized with 1N HCl. The resulting white precipitate was filtered and washed with water. The product 7 (0.8 g, 2.44 mmol, 87% yield) was dried under vacuum and was used for the next step without further purification. ¹H-NMR (DMSO-d₆/400 MHz): δ 11.68 (br, 1H); 7.39 (m, 5H); 6.35 (s, 1H); 3.97 (m, 2H); 3.40 (m, 4H); 2.50 (s, 3H); 2.08 (m, 6H); 1.52 (m, 2H).

Compound 1; N-(3-(trifluoromethyl)phenyl)-2-methyl-1-(3-morpholinopropyl)-5-phenyl-1H-pyrrole-3-carboxamide.

To a solution of 2-methyl-1-(3-morpholinopropyl)-5-phenyl-1H-pyrrole-3-carboxylic acid 7 (300 mg; 0.92 mmol) and 3-trifluoromethylaniline 8 (147 mg; 0.92 mmol) in 2 mL DMF was added EDCl (353 mg; 1.84 mmol), HOAt (250 mg; 1.84 mmol) and N-methyl morpholine (372 mg; 3.6 mmol). After stirring for 24 h at rt, the reaction mixture was quenched with water (20 mL), and the mixture was extracted with DCM. The organic phase was washed with water, brine, dried by anhydrous sodium sulfate, concentrated, and purified by chromatography to provide Compound 1 (0.260 g; 0.551 mmol, 60% yield). ¹H-NMR (CD₃OD/400 MHz): δ 8.11 (s, 1H); 7.86 (d, 1H); 7.26 (m, 7H); 6.65 (s, 1H); 4.06 (m, 2H); 3.53 (m, 4H); 2.64 (s, 3H); 2.14 (m, 6H); 1.63 (m, 2H). ¹⁹F-NMR (CD₃OD/376 MHz): −64.59 (s). MS (ES⁺, m/z): 472.1 (M⁺+1, 100.0).

An additional exemplary synthetic pathway for Compound 1 is outlined in Scheme 2. As the yield of the alkyation step in Scheme 1 was low, and the overall yield was only about 2.3%, an optimized synthetic route was developed with a significantly improved yield (70%). In the optimized route, the pyrrole ring formation and the alkylation of nitrogen in the pyrrole ring was carried out in one step by the reaction of 1,4-diketone 3 with 3-morpholinopropan-1-amine 9 in the presence of a catalytic amount of p-toluenesulfonic acid in refluxing EtOH. The alkylated intermediate 6 was obtained in qualitative yield. Also, the amidation step using acid chloride conditions (SOCl₂) provided a better yield of Compound 1 than with the EDCl/HOAt active ester, as well as an easier purification.

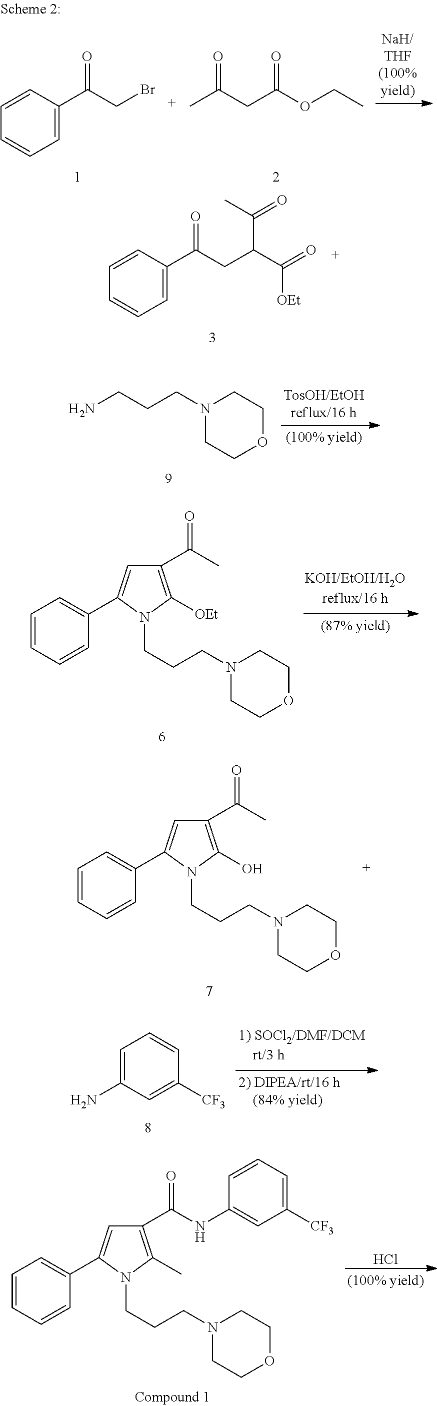

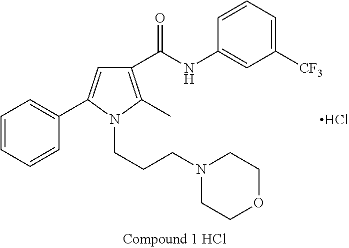 [SMILES: Cc1c(C(=O)Nc2cccc(C(F)(F)F)c2)cc(-c2ccccc2)n1CCCN1CCOCC1 ; Cl]

The compounds of Formulas I and II may be synthesized by the methods disclosed in Schemes 1 and 2. In an embodiment, a method of preparing the substituted N-(3-morpholinopropyl)-5-phenyl-1H-pyrrole-3-carboxamides of Formula II comprises reacting substituted 2-acetyl-4-oxo-4-phenyl butanoates with 3-morpholinopropan-1-amine, and reacting substituted N-(3-morpholinopropyl)-5-phenyl-1H-pyrrole-3-carboxylates with substituted anilines via an acid chloride.

Intermediate 6; ethyl 2-methyl-1-(3-morpholinopropyl)-5-phenyl-1H-pyrrole-3-carboxylate.

Toluene-4-sulphonic acid monohydrate (0.115 g; 0.61 mmol) was added under nitrogen to a solution of ethyl 2-acetyl-4-oxo-4-phenylbutanoate 3 (1.0 g; 4.03 mmol) and 3-morpholinopropan-1-amine 9 (0.58 g; 4.03 mmol) in ethanol (50 mL). The reaction mixture was refluxed for 16 h, then the solvent was evaporated. The crude product 6 (1.43 g; 4.03 mmol, 100% yield) was used for the next step without further purification. ¹H-NMR (CD₃OD/400 MHz): δ 7.42 (m, 2H); 7.35 (m, 3H); 6.42 (s, 1H); 4.23 (m, 2H); 4.04 (m, 2H); 3.53 (m, 4H); 2.60 (s, 3H); 2.14 (m, 6H); 1.61 (m, 2H), 1.33 (m, 3H). MS (ES⁺, m/z): 357.3 (M⁺-F1, 100.0).

Compound 1; N-(3-(trifluoromethyl)phenyl)-2-methyl-1-(3-morpholinopropyl)-5-phenyl-1H-pyrrole-3-carboxamide.

To a solution of 2-methyl-1-(3-morpholinopropyl)-5-phenyl-1H-pyrrole-3-carboxylic acid 7 (50 mg; 0.92 mmol) in 5 mL DCM was added thionyl chloride (56 μL; 0.76 mmol) and DMF (20 μL). After stirring for 3 h at rt, the reaction mixture was evaporated and dried under vacuum. To the residue was added DCM (3 mL), 3-trifluoromethylaniline 8 (37 mg; 0.23 mmol), DIPEA (116 μL) and the reaction mixture was stirred overnight. To the reaction mixture was added water, and the mixture was extracted with DCM. The organic phase was washed with water, brine, dried by anhydrous sodium sulfate, concentrated, and purified by chromatography to provide Compound 1 (60 mg; 0.127 mmol, 84% yield).

Compound 1-HCl, N-(3-(trifluoromethyl)phenyl)-2-methyl-1-(3-morpholinopropyl)-5-phenyl-1H-pyrrole-3-carboxamide hydrochloride.

Compound N-(3-(trifluoromethyl)phenyl)-2-methyl-1-(3-morpholinopropyl)-5-phenyl-1H-pyrrole-3-carboxamide (Compound 1) was dissolved in isopropyl alcohol (2 mL), and 1N HCl was added to adjust the pH to 5-6. The solution was frozen and lyophilized to provide Compound 1-HCl as a white powder. ¹H-NMR (CD₃OD/400 MHz): δ 8.12 (s, 1H); 7.85 (d, 1H); 7.47 (m, 7H); 6.72 (s, 1H); 4.15 (m, 2H); 3.97 (m, 2H); 3.66 (m, 2H); 3.25 (m, 2H); 2.99 (m, 4H); 2.67 (s, 3H); 1.96 (m, 2H). ¹⁹F-NMR (CD₃OD/376 MHz): −64.63 (s).

Compound 2; iso-propyl 3-(2-methyl-1-(3-morpholinopropyl)-5-phenyl-1H-pyrrole-3-carboxamido)benzoate.

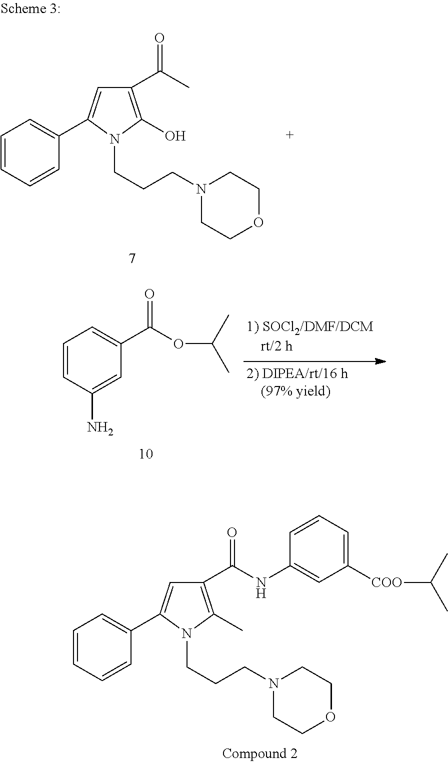

As shown in Scheme 3, Compound 2 was prepared in the following manner. To a solution of 2-methyl-1-(3-morpholinopropyl)-5-phenyl-1H-pyrrole-3-carboxylic acid 7 (200 mg; 0.610 mmol) in 5 mL DCM was added thionyl chloride (220 μL; 3.05 mmol) and DMF (20 μL). After stirring for 3 h at rt, the reaction mixture was evaporated and dried under vacuum. To the residue was added DCM (5 mL), isopropyl 3-aminobenzoate 10 (218 mg; 1.20 mmol), and DIPEA (0.42 mL), and the reaction mixture was stirred overnight. To the reaction mixture was added water, and the mixture was extracted with DCM. The organic phase was washed with water, brine, dried by anhydrous sodium sulfate, concentrated, and purified by chromatography to provide Compound 2 (290 mg; 0.592 mmol, 97% yield). ¹H-NMR (CD₃OD/400 MHz): δ 8.32 (s, 1H); 7.89 (d, 1H); 7.71 (d, 1H); 7.42 (m, 6H); 6.65 (s, 1H); 5.21 (m, 1H); 4.07 (m, 2H); 3.54 (m, 4H); 2.64 (s, 3H); 2.15 (m, 6H); 1.64 (m, 2H); 1.38 (d, 6H). MS (ES⁺, m/z): 490.1 (M⁺+1, 100.0).

Compound 2-HCl; iso-propyl 3-(2-methyl-1-(3-morpholinopropyl)-5-phenyl-1H-pyrrole-3-carboxamido)benzoate hydrochloride.

Compound 2 was dissolved in isopropyl alcohol (2 mL), and 1N HCl was added to adjust the pH to 5-6. The solution was frozen and lyophilized to provide Compound 2-HCl as a white powder. ¹H-NMR (DMSO-d₆/400 MHz): δ 9.65 (s, 1H); 8.35 (s, 1H); 8.07 (d, 1H); 7.62 (d, 1H); 7.45 (m, 5H); 6.89 (s, 1H); 5.15 (m, 1H); 4.01 (m, 2H); 3.92 (m, 2H); 3.65 (m, 2H); 3.28 (m, 2H); 2.97 (m, 4H); 2.65 (s, 3H); 1.93 (m, 2H), 1.32 (d, 6H).

Compound 3; 2-methyl-1-(3-morpholinopropyl)-5-phenyl-N-(3-(trifluoromethoxy)phenyl)-1H-pyrrole-3-carboxamide.

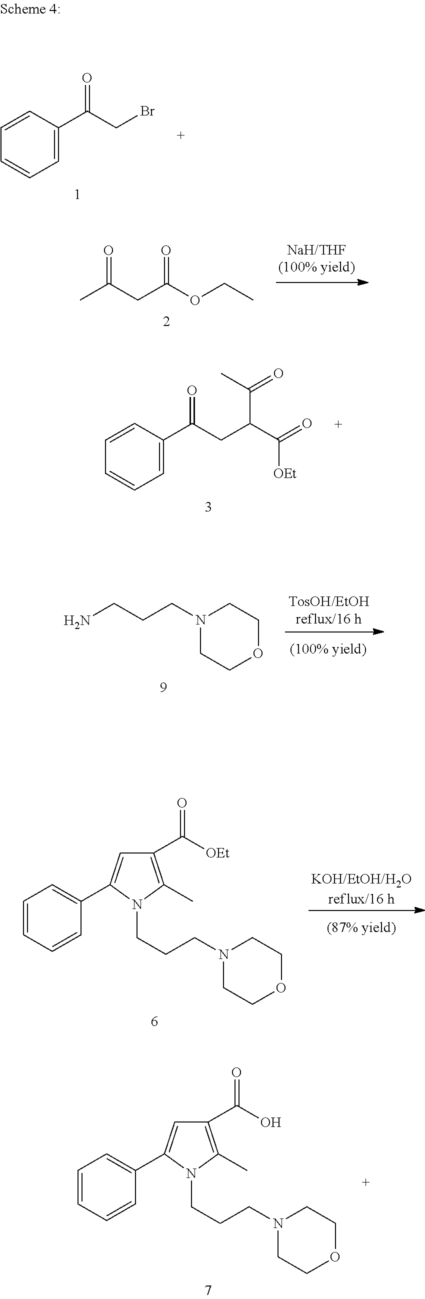

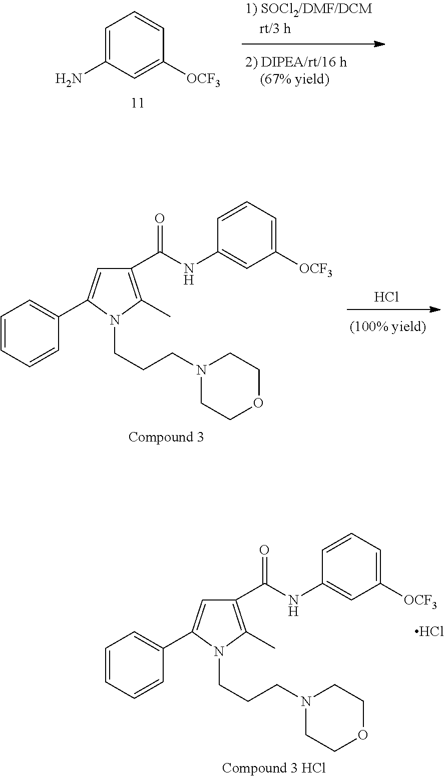 [SMILES: Nc1cccc(OC(F)(F)F)c1 ; Cc1c(C(=O)Nc2cccc(OC(F)(F)F)c2)cc(-c2ccccc2)n1CCCN1CCOCC1 ; Cl ; Cc1c(C(=O)Nc2cccc(OC(F)(F)F)c2)cc(-c2ccccc2)n1CCCN1CCOCC1 ; Cl]

As shown in Scheme 4, Compound 3 was prepared in the following manner. To a solution of 2-methyl-1-(3-morpholinopropyl)-5-phenyl-1H-pyrrole-3-carboxylic acid 7 (100 mg; 0.305 mmol) in 5 mL DCM was added thionyl chloride (111 μL; 1.53 mmol) and DMF (20 μL). After stirring for 3 h at rt, the reaction mixture was evaporated and dried under vacuum. To the residue was added DCM (3 mL), 3-trifluoromethoxyaniline 11 (108 mg; 0.61 mmol), and DIPEA (0.22 mL; 1.2 mmol), and the reaction mixture was stirred overnight. To the reaction mixture was added water, and the mixture was extracted with DCM. The organic phase was washed with water, brine, dried by anhydrous sodium sulfate, concentrated, and purified by chromatography to provide Compound 3 (99 mg; 0.203 mmol, 67% yield). ¹H-NMR (CD₃OD/400 MHz): δ 7.80 (s, 1H); 7.58 (d, 1H); 7.40 (m, 6H); 6.96 (d, 1H); 6.65 (s, 1H); 4.08 (m, 2H); 3.57 (m, 4H); 2.64 (s, 3H); 2.30 (m, 6H); 1.67 (m, 2H). ¹⁹F-NMR (CD₃OD/376 MHz): −59.73 (s). MS (ES⁺, m/z): 488.0 (M⁺+1, 100.0).

Compound 3-HCl, 2-methyl-1-(3-morpholinopropyl)-5-phenyl-N-(3-(trifluoromethoxy)phenyl)-1H-pyrrole-3-carboxamide hydrochloride.

Compound 3 was dissolved in isopropyl alcohol (2 mL), and 1N HCl was added to adjust the pH to 5-6. The solution was frozen and lyophilized to Compound 3-HCl as a white powder. ¹H-NMR (DMSO-d₆/400 MHz): δ 9.69 (s, 1H); 7.94 (s, 1H); 7.93 (d, 1H); 7.34 (m, 6H); 7.00 (d, 1H); 6.85 (s, 1H); 4.00 (m, 2H); 3.91 (m, 2H); 3.68 (m, 2H); 3.28 (m, 2H); 2.64 (s, 3H); 2.50 (m, 4H); 1.94 (m, 2H). ¹⁹F-NMR (CD₃OD/376 MHz): −56.99 (s).

Compound 4; 2-methyl-1-(3-morpholinopropyl)-5-phenyl-1H-pyrrole-3-carboxylic acid, 1-hydroxy-7-azabenzotriazol-1-yl ester.

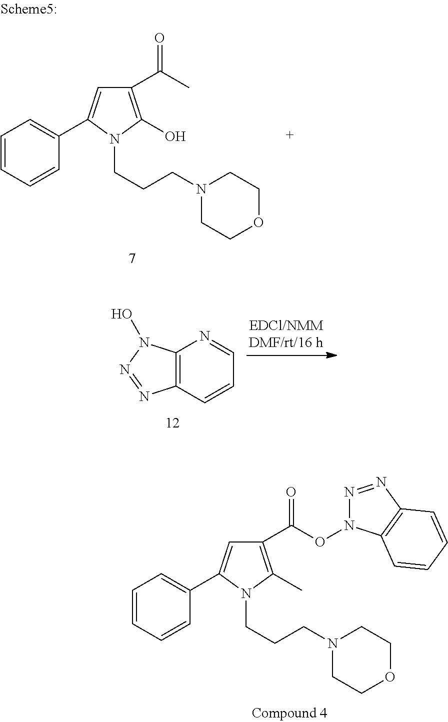 [SMILES: CC(=O)c1cc(-c2ccccc2)n(CCCN2CCOCC2)c1O ; On1nnc2cccnc21 ; Cc1c(C(=O)On2nnc3ccccc32)cc(-c2ccccc2)n1CCCN1CCOCC1]

As shown in Scheme 5, Compound 4 was prepared in the following manner. To a solution of 2-methyl-1-(3-morpholinopropyl)-5-phenyl-1H-pyrrole-3-carboxylic acid 7 (300 mg; 0.92 mmol) and 1-hydroxy-7-azabenzotriazole (250 mg; 1.84 mmol) in 2 mL DMF was added EDCl (353 mgl 1.84 mmol) and N-methyl morpholine (372 mgl 3.6 mmol). After stirring for 24 h at rt, the reaction mixture was quenched with water (20 mL), and the mixture was extracted with DCM. The organic phase was washed with water, brine, dried by anhydrous sodium sulfate, concentrated, and purified by chromatography to provide Compound 4. ¹H-NMR (CD₃OD/400 MHz): δ 8.75 (d, 1H); 8.54 (d, 1H); 7.58 (m, 1H); 7.48 (m, 6H); 6.81 (s, 1H); 4.15 (m, 2H); 3.55 (m, 4H); 2.66 (s, 3H); 2.22 (m, 6H); 1.68 (m, 2H). MS (ES⁺, m/z): 447.1 (M⁺+1, 100.0).

Compound 5; N-(3-(trifluoromethyl)phenyl)-2-methyl-1-(3-morpholinopropyl)-1H-indole-3-carboxamide.

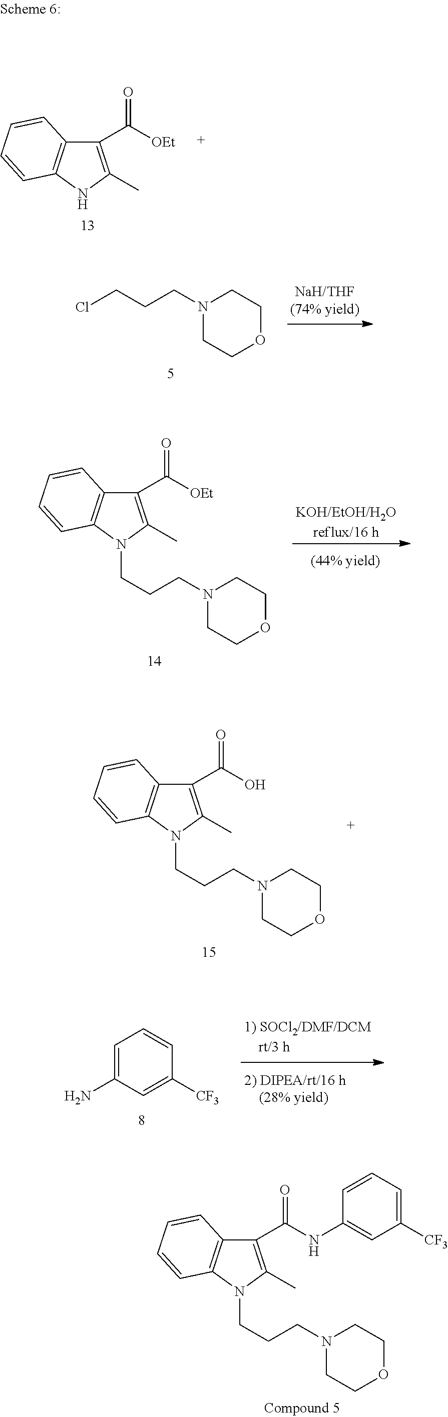

Intermediate 14; ethyl 2-methyl-1-(3-morpholinopropyl)-1H-indole-3-carboxylate.

As shown in Scheme 6, Compound 5 was prepared in the following manner. A solution of ethyl 2-methyl-1H-indole-3-carboxylate 13 (250 mg, 1.23 mmol) in DMF (3 mL) was treated with NaH in mineral oil (34 mg, 1.50 mmol) and the mixture was stirred at rt for 10 min. Following the addition of 4-(3-chloropropyl)morpholine 5, the mixture was stirred at 50° C. for 24 h. After cooling to rt, the reaction mixture was quenched with water (20 mL). The reaction mixture was extracted with ethyl acetate. Purification by silica gel chromatography provided product 14 (300 mg; 0.909 mmol, 74% yield). ¹H-NMR (CDCl₃/400 MHz): δ 8.11 (m, 1H), 7.34 (m, 1H), 7.25 (m, 2H), 4.38 (m, 2H), 4.25 (m, 2H), 3.83 (m, 4H), 2.78 (s, 3H), 2.50 (m, 6H), 2.11 (m, 2H), 1.44 (m, 3H).

Intermediate 15; 2-methyl-1-(3-morpholinopropyl)-1H-indole-3-carboxylic acid.

To a solution of ethyl 2-methyl-1-(3-morpholinopropyl)-1H-indole-3-carboxylate 14 (300 mg; 0.909 mmol) in EtOH (5 mL) and water (1 mL), was added KOH (0.509 g; 9.09 mmol) at rt. The reaction mixture was refluxed overnight. EtOH was evaporated under reduced pressure. Water (10 mL) was added and the solution was neutralized with 1N HCl. The resulting white precipitate was filtered and washed with water. The product 15 (0.12 g, 0.40 mmol, 44% yield) was dried under vacuum and was used for the next step without further purification.

Compound 5; N-(3-(trifluoromethyl)phenyl)-2-methyl-1-(3-morpholinopropyl)-1H-indole-3-carboxamide.

To a solution 2-methyl-1-(3-morpholinopropyl)-1H-indole-3-carboxylic acid 15 (120 mg; 0.40 mmol) in 5 mL DCM was added thionyl chloride (150 μL; 2.0 mmol) and DMF (20 μL). After stirring for 3 hours at rt, the reaction mixture was evaporated and dried under vacuum. To the residue was added DCM (3 mL), 3-trifluoromethoxylaniline 8 (129 mg; 0.80 mmol), and DIPEA (0.30 mL; 1.6 mmol), and the reaction mixture was stirred overnight. To the reaction mixture was added water, and the mixture was extracted with DCM. The organic phase was washed with water, brine, dried by anhydrous sodium sulfate, concentrated, and purified by chromatography to provide Compound 5 (50 mg; 0.112 mmol, 28% yield). ¹H-NMR (CD₃OD/400 MHz): δ 8.15 (s, 1H); 7.86 (d, 1H); 7.77 (d, 1H); 7.51 (m, 2H); 7.39 (m, 1H); 7.17 (m, 2H); 4.26 (m, 2H); 3.65 (m, 4H); 2.68 (s, 3H); 2.33 (m, 6H); 1.93 (m, 2H). ¹⁹F-NMR (CD₃OD/376 MHz): −64.52 (s). MS (ES⁺, m/z): 446.1 (M⁺+1, 100.0).

Protected derivatives of the disclosed compounds also are contemplated. A variety of suitable protecting groups for use with the inventive compounds are disclosed. Other conventional protecting groups can be selected by those of skill in the art in consultation with Greene and Wuts, Protective Groups in Organic Synthesis; 3rd Ed.; John Wiley & Sons, New York, 1999.

The synthetic schemes and methods disclosed herein may be readily applied to the synthesis of additional compounds containing a pyrrole or indole core structure, as recognized by those skilled in the art.

### III. PHARMACEUTICAL COMPOSITIONS

The compounds of Formula I disclosed herein may be included in pharmaceutical compositions, including therapeutic and prophylactic formulations. These compositions may be combined together with one or more pharmaceutically acceptable vehicles, excipients or carriers and, optionally, other therapeutic ingredients (including, for example, antibiotics, anti-inflammatory agents, anesthetics, steroids, carbonic anhydrase inhibitors, beta-adrenergic receptor antagonists, vasodilators and anti-viral agents). In an embodiment, the composition may further comprise at least one of timolol, dexamethasone, prednisone, brimonidine, dorzolamide, travoprost, bimatoprost, pilocarpine and lantanoprost. The compositions disclosed herein may be combined with or used in combination with other ocular therapies, as described herein.

In an embodiment, the pharmaceutical composition comprises a compound of claim 1 and at least one excipient.

The compounds and pharmaceutical compositions described herein may be administered topically, periocularly or intraocularly. For example, the pharmaceutical compositions may be injected into or adjacent to a subjects' eye, such as via a subconjunctival, retrobulbar, juxtascleral or intravitreal injection. An ophthalmic device such as an implant may also be used to deliver the composition. The treatment of chronic ocular diseases, such as glaucoma, may be particularly amenable to a topically applied composition, such as an eyedrop. In certain embodiments, the pharmaceutical composition comprises a gel-forming solution. In some embodiments, the compounds of Formula I are administered via an intraocular injection.

To formulate the pharmaceutical compositions, a compound of Formula I may be combined with various pharmaceutically acceptable additives, as well as a base or vehicle for dispersion of the compound. Such additives include, but are not limited to, pH control agents (for example, arginine, sodium hydroxide, glycine, hydrochloric acid, citric acid), local anesthetics (for example, benzyl alcohol), isotonizing agents (for example, sodium chloride, mannitol, sorbitol), adsorption inhibitors (for example, Tween 80 or medium chain triacylglycerols such as myglyol 812), solubility enhancing agents (for example, cyclodextrins and derivatives thereof), stabilizers (for example, serum albumin), and reducing agents (for example, glutathione). An antimicrobial agent may also be added.

Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Mont.) and IL-12 (Genetics Institute, Cambridge, Mass.), among many other suitable adjuvants well known in the art, may be included in the composition. When the composition is a liquid, the tonicity of the formulation, as measured with reference to the tonicity of 0.9% (w/v) physiological saline solution taken as unity (e.g., isotonic), may be adjusted to a value at which no substantial, irreversible tissue damage will be induced at the site of administration. In an embodiment, it may be desirable to include isotonic agents, for example, sugars, polyalcohols such as mannitol and sorbitol, or sodium chloride in the composition, to achieve an isotonic composition. In certain embodiments, non-isotonic compositions may be desirable.

A compound may be dispersed in a base or vehicle, which can include a hydrophilic compound having a capacity to disperse the compound, and any additives. The base may be selected from a wide range of suitable compounds, including but not limited to, copolymers of polycarboxylic acids or salts thereof; carboxylic anhydrides (for example, maleic anhydride); with other monomers (for example, methyl(meth)acrylate and acrylic acid); hydrophilic vinyl polymers, such as polyvinyl acetate, polyvinyl alcohol, polyvinylpyrrolidone, cellulose derivatives such as hydroxymethylcellulose and hydroxypropylcellulose; natural polymers, such as chitosan, collagen, sodium alginate, gelatin, hyaluronic acid; and nontoxic metal salts thereof.

A biodegradable polymer may be selected as a base or vehicle, such as, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters and sucrose fatty acid esters may be employed as vehicles. Hydrophilic polymers and other vehicles can be used alone or in combination, and enhanced structural integrity can be imparted to the vehicle by, for example, partial crystallization, ionic bonding, or cross-linking. The vehicle may be provided in a variety of forms, including fluid or viscous solutions, gels, pastes, powders, microspheres, and films for direct application to a mucosal surface.

The physical characteristics of the compounds of Formula I may aid in determining the appropriate composition for the treatment of a specific ocular disease. A lipophilic compound will generally be absorbed readily into the lipophilic corneal epithelium, whereas an ionic or hydrophilic compound will be absorbed more slowly.

The compound may be combined with the base or vehicle according to a variety of methods, and release of the compound may be, for example, via diffusion or disintegration of the vehicle. In some embodiments, the compound may be dispersed in an ocular implant or insert. The implant or insert may be designed to degrade in the presence of tear fluid.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity.

Exemplary polymers include polyglycolic acids and polylactic acids, poly(DL-lactic acid-co-glycolic acid), poly(D-lactic acid-co-glycolic acid), and poly(L-lactic acid-coglycolic acid). Other useful biodegradable or bioerodable polymers include, but are not limited to, poly(epsilon-caprolactone), poly(epsilon-caprolactone-CO-lactic acid), poly(epsilon-caprolactone-CO-glycolic acid), poly(beta-hydroxy butyric acid), poly(alkyl-2-cyanoacrylate), hydrogels such as poly(hydroxyethyl methacrylate), polyamides, poly(amino acids) such as L-leucine, glutamic acid, L-aspartic acid, poly(ester urea), poly(2-hydroxyethyl DL-aspartamide), polyacetal polymers, polyorthoesters, polycarbonate, polymaleamides, polysaccharides, and copolymers thereof.

Pharmaceutical compositions for administering the compound may also be formulated as gel-forming solutions. For example, a composition may be a low-viscosity solution in the delivery container but gels upon contact with the tear fluid. This type of formulation can provide an increased rate of drug absorption and a prolonged duration of the therapeutic effect. In certain embodiments, the compositions may include gellan or xanthan gum.

The pharmaceutical composition may be configured to provide the compound of Formula I to the eye for a short duration, such as seconds, or for months to years. Methods for preparing such formulations are known to those skilled in the art (see, for example, Sustained and Controlled Release Drug Delivery Systems, J. R. Robinson, ed., Marcel Dekker, Inc., New York, 1978; and Remington: The Science and Practice of Pharmacy (21st Edition), Lippincott Williams & Wilkins, Maryland, 2006; both incorporated by reference herein). In certain embodiments, the composition is applied once-daily, or qd. In some embodiments, the composition may be applied twice a day (bid), three times a day (tid) or four times a day (qid).

The pharmaceutical compositions of the disclosure typically are sterile and stable under the conditions of manufacture, storage and use. Sterile solutions can be prepared by incorporating the compound in the required amount in an appropriate solvent with one or a combination of ingredients enumerated herein, as required, followed by filtered sterilization. Dispersions may be prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antimicrobial, antibacterial and/or antifungal agents, for example, quaternary ammonium compounds such as BAC, oxidizing agents such as sodium perborate, the parabens, chlorobutanol, and thimerosal.

Determination of effective dosages in this context may be based on animal model studies followed by human clinical trials and may be guided by administration protocols that significantly reduce the occurrence or severity of targeted disease symptoms or conditions in the subject. Suitable models in this regard include, for example, murine, rat, avian, porcine, feline, non-human primate, and other accepted animal model subjects known in the art. Alternatively, effective dosages may be determined using in vitro models (for example, immunologic and histopathologic assays). Using such models, calculations and adjustments may be required to determine an appropriate concentration and dose to administer a therapeutically effective amount of the compound (for example, amounts that are effective to alleviate one or more symptoms of a targeted disease, such as to normalize IOP). In certain embodiments, an effective amount or effective dose of the compound may simply inhibit or enhance one or more selected biological activities correlated with a disease or condition, as set forth herein, for either therapeutic or diagnostic purposes.

The actual dosage of the compound may vary according to factors such as the disease indication and particular status of the subject (for example, the subject's age, size, fitness, extent of symptoms, and susceptibility factors), time and route of administration, other drugs or treatments being administered concurrently, as well as the specific pharmacology of the compound for eliciting the desired activity or biological response in the subject. Dosage regimens can be adjusted to provide an optimum prophylactic or therapeutic response.

A therapeutically effective amount may be one in which any toxic or detrimental side effects of the compound and/or other biologically active agent is outweighed in clinical terms by therapeutically beneficial effects. A non-limiting range for a therapeutically effective amount of a compound and/or other biologically active agent within the methods and compositions of the disclosure is about 0.01 mg/kg body weight to about 100 mg/kg body weight, such as about 0.05 mg/kg to about 50 mg/kg body weight, or about 0.5 mg/kg to about 5 mg/kg body weight.

The dosage may be varied to maintain a desired concentration in the eye. Higher or lower concentrations can be selected based on the mode of delivery, for example, topical delivery versus intraocular delivery. Dosage can also be adjusted based on the release rate of the administered formulation, for example, of a topical formulation versus an intraocular injection formulation.

The instant disclosure also includes kits, packages and multi-container units containing the herein described pharmaceutical compositions, active ingredients, and/or devices and consumables that facilitate the administration the same for use in the prevention and treatment of diseases and other conditions in mammalian subjects. For example, the kit may contain an injector configured for intraocular use.

In an embodiment, the compound of Formula I may be formulated in a pharmaceutical preparation for delivery to a subject. The compound may be contained in a bulk dispensing container or unit or multiunit dosage form. Optional dispensing means can be provided, for example, an intraocular injector. Packaging materials optionally include a label or instruction indicating for what treatment purposes and/or in what manner the pharmaceutical agent packaged therewith can be used.

### IV. METHODS OF TREATMENT

The compounds and pharmaceutical compositions disclosed herein may be used for treating or preventing ocular diseases, such as retinopathies including non-proliferative and proliferative diabetic retinopathy and retinopathy of prematurity, glaucoma, macular degeneration, age-related macular degeneration (wet and dry), retinitis pigmentosa, Stargardt disease, macular edema, uveitis, and retinal infections including those with cytomegalovirus. The disease may be chronic, or it may be acute. The compounds and pharmaceutical compositions disclosed herein may also be used to prevent blast-induced ocular injury mediated by, for example, IEDs, increased G forces during flight and mechanical trauma. In an embodiment, the compounds and pharmaceutical compositions disclosed herein may be used for treating or preventing glaucoma.

It is possible that the vertebrate retina, which is exposed to systemic blood pressure, hydrostatic pressure form the CSF, and intrinsic IOP, contains one or more pressure-sensitive TRP and/or piezo channels. Pathological elevations in IOP or systemic pressure represent primary risk factors for many conditions such as glaucoma, a group of inherited optic neuropathies characterized by apoptotic loss of RGCs, degeneration of the optic nerve, and progressive loss of visual fields. The cellular pathophysiology of glaucoma is not well understood, in part because the mechanisms that couple the mechanical stimulus (ΔIOP) to cellular signal transduction remain to be characterized.

The compounds and compositions disclosed herein may be useful for the treatment or prevention of diseases and disorders other than ocular diseases, for which an antagonist of a TRPV4 channel may be beneficial. For example, the compounds of Formula I may be useful in the treatment and/or prevention of disorders of the bladder, pulmonary diseases including heart failure, and lung edema.

Additionally, various ocular traumas are capable of having long-term degenerative effects on the eye. For example, a subject that is near an explosion can develop subsequent degenerative conditions due to the compressive impact of the explosion on the eyes. In some cases such damage may not be immediate, but can develop over time. Treatments for such ocular trauma can include delivering a TRPV4 antagonist into the eye following such trauma to moderate, decrease, or eliminate associated long-term effects.

Furthermore, in some cases apoptosis of RGCs can be associated with ocular conditions having a degenerative component. In some aspects, antagonists to TRPV4 receptors can have a neuroprotective effect on at least RGCs, thus treating or moderating the damaging effects of such conditions. In one specific aspect, a method for treating an ocular condition associated with apoptotic RGC death is provided. Such a method can include delivering a TRPV4 antagonist into an eye of a subject such that the TRPV4 antagonist protects the retinal ganglion cells from apoptotic cell death. It is noted that treatment of an eye with a TRPV4 antagonist can be beneficial for those subjects experiencing and increase in IOP as well as for those subjects that do not exhibit IOP or have moderate increases in IOP.

Traditional glaucoma therapies are designed to lower IOP in the anterior eye through eye drops that decrease the rate of fluid production (β-adrenoceptor antagonists, α1-adrenoceptor agonists and carbonic acid inhibitors) or increase fluid outflow in the anterior eye (prostaglandin F2a analogs and muscarinic agonists). These interventions are far from ideal because these drugs do not protect RGCs, there is a significant cohort of nonresponding patients and there are side effects. Patients with “low-tension” glaucoma (30-40% total) develop the disease at ‘normal’ IOP yet still respond to IOP lowering drugs, suggesting their RGC targets are excessively sensitive to pressure. Thus neuroprotection of at least RGCs can be beneficial in the treatment of various ocular conditions, such as for example, glaucoma.

In one aspect, a TRPV4 antagonist can provide protection against pressure-induced Ca++ overloads and RGC death under in vitro and in vivo conditions. Such an 10 antagonist reduces IOP in the anterior chamber of eye, suggesting that it regulates fluid production/absorption in the trabecular meshwork of the anterior eye, and blocks pressure-induced apoptosis of RGCs.

It is known that the TRPV4 ion channel is involved in modulating calcium flux and apoptosis of murine RGCs, and is implicated in the retinal remodeling that occurs during chronic increases in IOP (Ryskamp et al, J. Neuroscience 2011, 31(19), 7089-7101, incorporated herein). Increases in hydrostatic or IOP are correlated with RGC death in cell culture, mouse models and human glaucoma patients, and lowering of the IOP slows the progression of axonal loss in glaucomateous degeneration (id). Disclosed herein is evidence that the compounds of Formula I can antagonize excessive TRPV4 activation and thus not only lower IOP, but also be protective against the apoptosis in RGCs which results from chronic mechanical and/or osmotic stimulation. The regulation of IOP by modulating fluid production in the anterior eye and neuronal cell loss in the posterior eye is a novel dual approach for treating ocular diseases, particularly glaucoma.

The specific examples included herein are for illustrative purposes only and are not to be considered as limiting to this disclosure. Any active agents and reagents used in the following examples are either commercially available or can be prepared according to standard literature procedures by those skilled in the art. In light of this disclosure, those of skill in the art will recognize that variations of these examples and other examples of the disclosed method would be possible without undue experimentation.

### Example 1

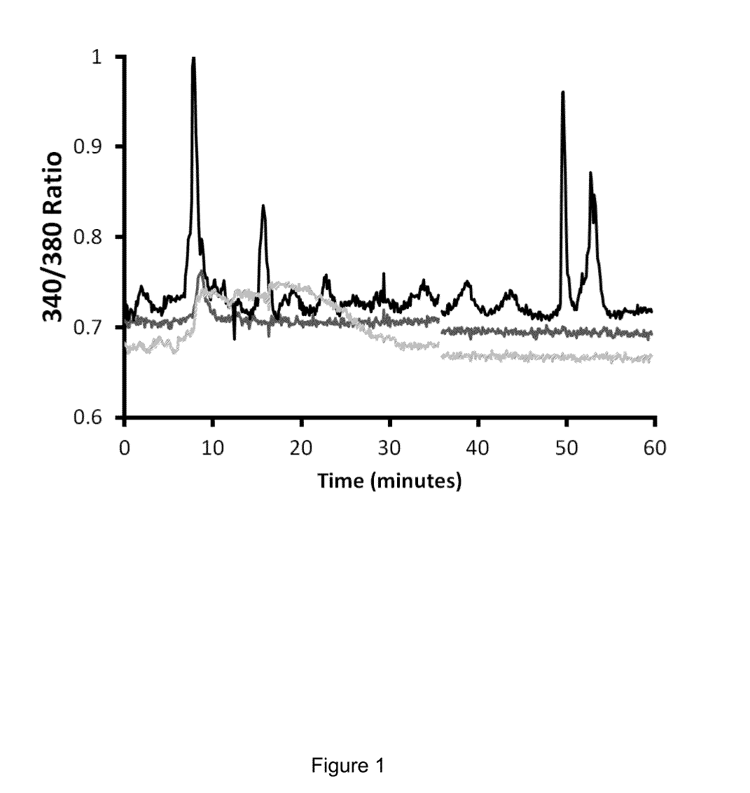

Intracellular calcium was measured to demonstrate that TRPV4 is functional in cells of the human trabecular meshwork, as shown in FIG. 1. Pieces of tissue were isolated from a trabeculectomy. Trabecular meshwork cells (TM) were kept in cold L15 medium and then loaded with Fura-2 (10 μm) for 1 hour. Fibroblast-shaped cells were imaged from a TM chunk (˜500 μm×500 μm) with a 40× water immersion objective. The sampling rate was at 6 second intervals. Krebs Ringer Bicarbonate Recording buffer (7.4, 284 mOsm) was prepared as reported by Anthony et al., 1998, IOVS; Prostaglandin F2α receptors in the human trabecular meshwork.

Calcium imaging was performed on TM cells isolated from human patients. In brief, dissociated human TM cells embedded within the tissue were dissociated and plated on concanavalin A-coated (0.2 mg/ml; Sigma) coverslips, loaded with fura-2 AM (1-510 μM; Invitrogen Life Technologies) for 15 min-1 hour and washed for 10 min in dye-free L-15 medium. Cells were viewed with Nikon Ti inverted or 600EF upright microscopes using 20×0.95 numerical aperture (NA), 40×0.85 NA, or 40×1.25 NA objective lenses. Excitation for 340 and 380 nm filters (Chroma and Semrock) was provided by a 150W Xenon are lamp (DG4, Sutter Instruments). Fluorescence emission was high-pass filtered at 510 nm and captured with cooled digital CCD cameras (HQ2, Photometrics). Data acquisition and F340/F380 ratio calculations were performed by NIS Elements software. Fluorescence imaging was performed on regions of interest (ROIs) encompassing the TM cell somata, typically at 3×3 binning. Background fluorescence was measured in similarly sized ROIs in neighboring areas devoid of cells. After sequential image acquisition (0.167-0.5 Hz) of cell fluorescence at 340/380 nm, the background was subtracted.

Glutamate (100 μM) was added at the beginning of each experiment to control for cell health, type, and responsiveness. DMSO, the solvent for the indicator dye, did not induce any responses (data not shown). Experiments were conducted at room temperature. Bath application of the TRPV4 agonist GSK1016790A (hereinafter “GSK” or “GSK101”) (100 nM) indicated by black bars resulted in increased 340/380 fluorescence ratio, consistent with TRPV4-mediated increases in the intracellular calcium concentration [Ca++]ᵢ. These data show that human TM cells express functional TRPV4 channels.

### Example 2

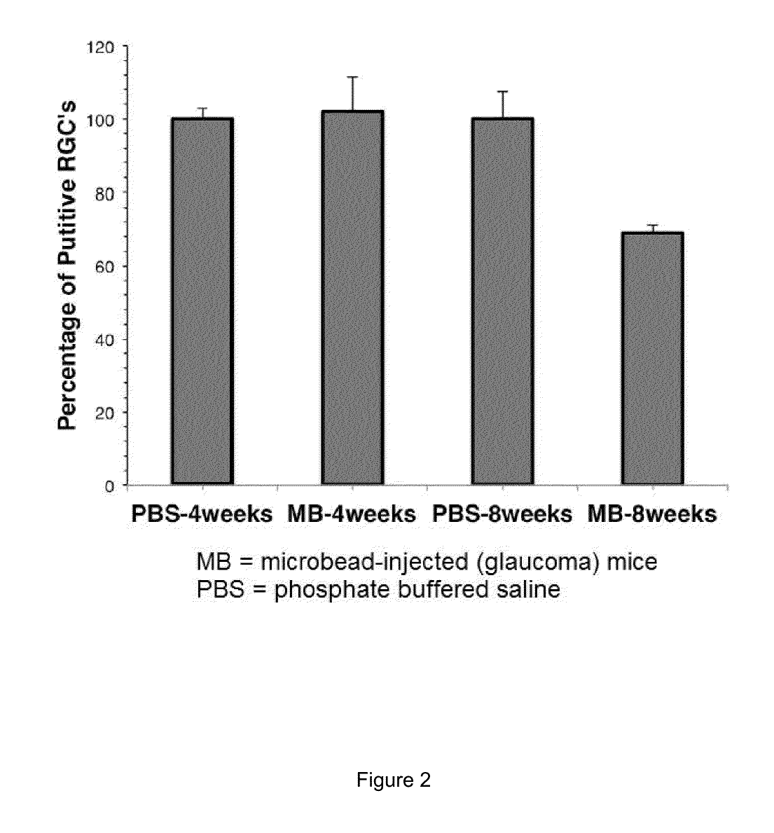

FIG. 2 confirms that the mouse model for glaucoma causes RGC degeneration. Specifically, microbeads (MBs) were injected into the anterior chamber of a mouse eye. The MBs increase intraocular IOP above 15 mm Hg by blocking outflow of the aqueous humor within the anterior chamber of the eye. MBs were reinjected as needed to maintain a high IOP, typically one injection every two weeks. Contralateral eyes were injected intraocularly with the vehicle PBS (phosphate buffered saline) as controls. No pressure increase was observed in PBS-injected eyes. IOP levels were measured at least twice weekly before and after intraocular injections. After injections, mice recovered for 48 hours before IOP measurements resumed. Following 4 or 8 weeks of IOP elevation, mice were intracardially perfused with 4% paraformaldehyde in 1×PBS to fix the retinas. Retinas were then removed and processed for immunohistochemistry (IHC). Whole retinas were rinsed with 1×PBS three times at room temperature then incubated in blocking buffer (1×PBS+1% BSA). Blocking buffer was removed and PBS containing primary antibody diluted in PBS was added to retinas and incubated overnight at 4° C. After rinsing with PBS, PBS containing the secondary antibody conjugated to fluorophores (Alexa 488 or Alexa 594; Life Technologies) and diluted in PBS at a concentration of 1:1000 was added and incubated for 1 hour at room temperature. Retinas were rinsed and flat-mounted on microscope slides. Mounting medium was added and retinal wholemounts were coverslipped. The density of RGCs was imaged with a confocal microscope (Zeiss LSM 510) and quantified by counting NeuN-, CFP- and/or Tuj-1—labeled somata in the RGC layer of the retinal wholemount preparation. Injection of microbeads induces RGC degeneration after 8 weeks but not after 4 weeks.

### Example 3

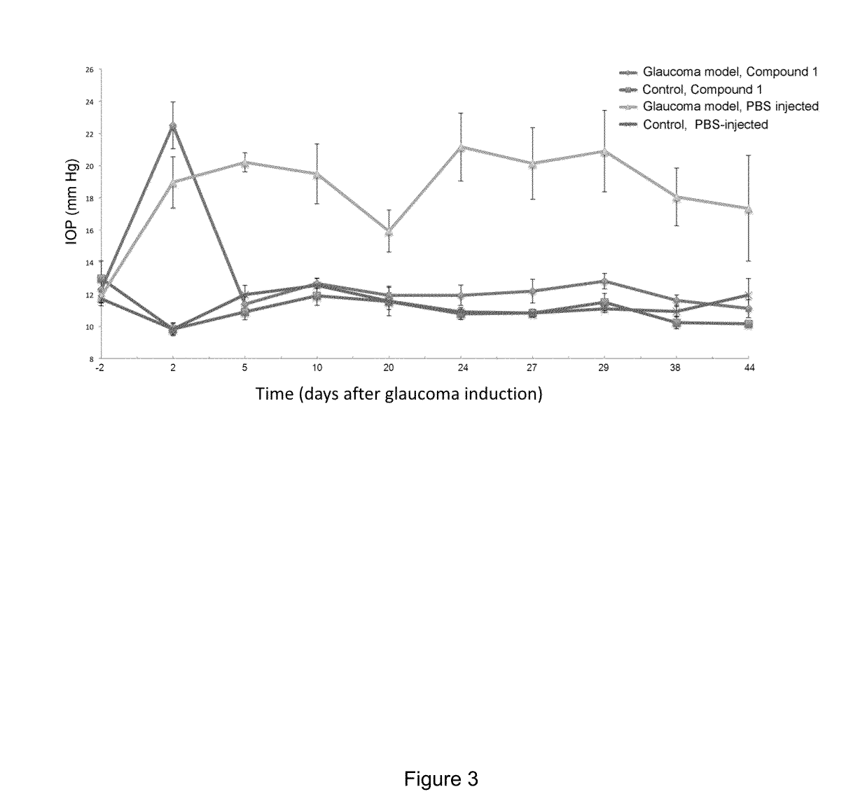

FIG. 3 demonstrates that an exemplary TRPV4 antagonist as disclosed herein rescues the eye from elevated IOP in the mouse model described in FIG. 2. Specifically, microbeads (MBs) were injected into the anterior chamber of a mouse eye as described in Example 2 with PBS-injected eyes used as controls. Compound 1 (10 mg/kg body weight) (or PBS vehicle) was injected intraperitoneally (IP). FIG. 3 shows that MBs increased IOP in glaucoma mice. In contrast, Compound 1 blocked the increase in IOP to the level observed in control mice (PBS injected eyes). IOP was measured in mice between 10:00 AM and 1:00 PM with the TonoLab rebound tonometer (Colonial Medical Supply, Franconia, NH/Tyolat, Helsinki, Finland). Mice were sedated with IP injection of Avertin with final amount calculated by weight (e.g., 0.5 ml for 21-24 g animals). Animals were placed on a jack stand platform and the tonolab was clamped on a ring stand and centered onto the mid-cornea. During measurements animal were neither restrained nor touched. Each eye was measured twenty consecutive times, the highest and lowest values were discarded and the values were averaged. FIG. 3 shows that MBs increased IOP in the glaucoma model (MG-injected mice). In contrast, Compound 1 blocked the increase in IOP to the level observed in control mice (PBS injected eyes).

### Example 4

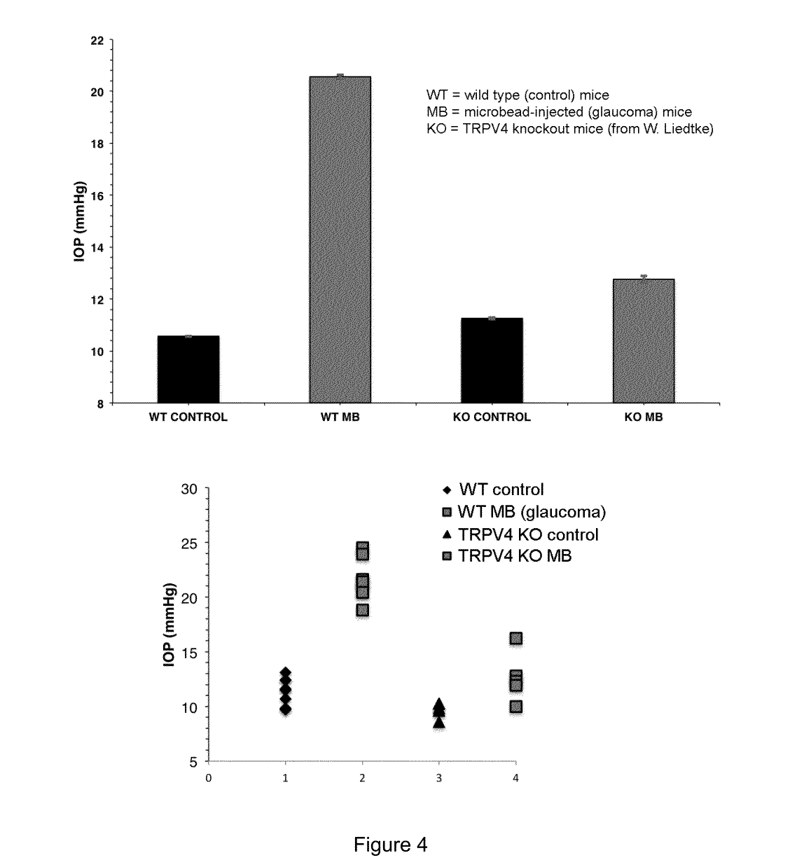

The experiment disclosed in Example 4 demonstrates that TRPV4 is required for the increased IOP observed in the MB model of glaucoma. The anterior chambers of TRPV4-null mice and wild type controls were injected with MBs or PBS as described in Example 2. IOP was measured as in Example 3. While the MBs caused increased IOP in wild type control mice, they did not change IOP in TRPV4-null mice. FIG. 4 illustrates these data. The top graph represents average IOP between individuals and the bottom graph illustrates IOP from representative individual micemice.

### Example 5

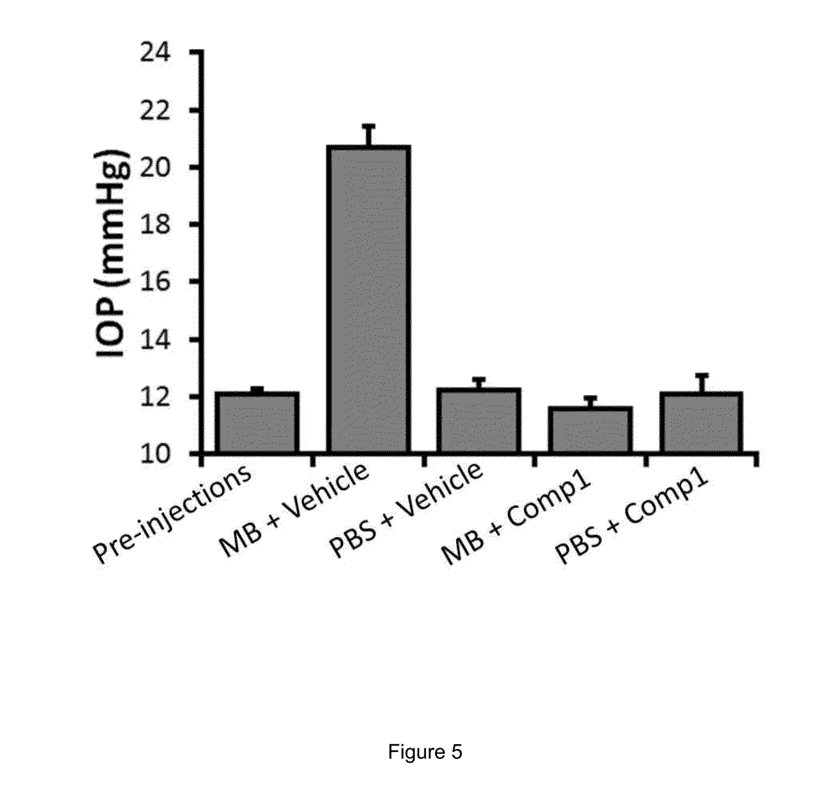

FIG. 5 shows the cumulative effectiveness of IP-injected Compound 1, with the average amount of IOP that occurs in the presence and absence of Compound 1, an exemplary TRPV4 antagonist, in the MB mouse model of glaucoma. Each bar represents cumulative data from 3 independent experiments each consisting of 4 cohorts: (1) animals injected intraocularly with MBs and receiving daily IP injections of Compound 1; (2) animals injected intraocularly with the vehicle (PBS) and receiving daily IP injections of PBS; (3) animals injected intraocularly with MBs and receiving daily IP injections of Compound 1; and (4) animals, injected intraocularly with PBS and receiving daily IP injections of Compound 1. Every cohort consisted of 15-20 mice. These data shows that Compound 1 blocks MB-induced increases in IOP.

### Example 6

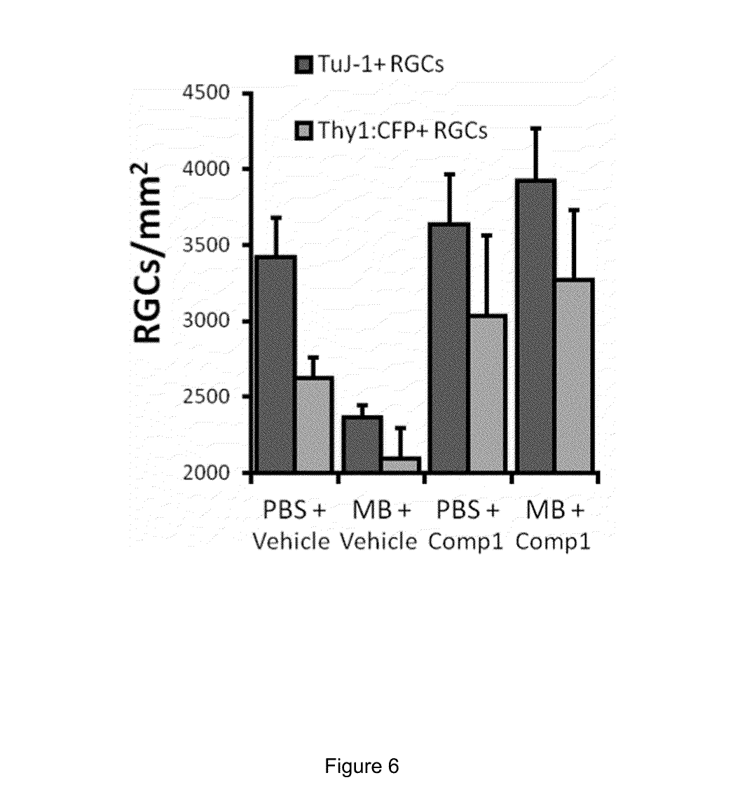

FIG. 6 shows the relationship of RGC death and IOP in the presence and absence of an exemplary TRPV4 antagonist. RGD density was quantified in retinas isolated from TuJ-1⁺ mice and transgenic Thy1-CRP⁺ transgenic mice that had been injected with either MBs or vehicle (PBS) then dosed with either compound 1 or vehicle (PBS). TuJ-1⁺ cells are those that express the retinal ganglion cell marker, tubulin III (TuJ-1⁺) whereas CFP is a fluorescent marker expressed in a subset of retinal ganglion cells of the Thy1:CFP mouse strain. TuJ-1 was quantified by immunohistochemistry using a Tuj-1-specific antibody conjugated to a fluorescent Alexa 488 or Alexa 594 secondary antibody. For immunohistochemistry, eyes were enucleated and their corneas and lenses dissected away. The remaining posterior pole of the eye was fixed by immersion for 1 hour at room temperature in freshly prepared 4% paraformaldehyde in 0.1 M phosphate buffer, pH 7.2, then washed 3×10 min. in pH 7.2 phosphate buffered saline (PBS). Fixed retinas were then immersed for 12-16 hours in 30% sucrose at 4° C. then embedded in OCT (Ted Pella, Redding, Pa.). For fluorescence immunocytochemistry, retinal wholemounts were washed in PBS, then placed for 30 min. to 1 hour in blocking solution (10 ml PBS, 30 μL Triton-X 100, 100 mg bovine serum albumin, 100 μL 10% w/v Na azide solution) in OCT (Ted Pella, Redding, Pa.). For fluorescence immunocytochemistry, retinal wholemounts were washed in PBS, then placed for 30 min. to 1 hour in blocking solution (10 ml PBS, 30 μL Triton-X 100, 100 mg bovine serum albumin, 100 μL 10% w/v Na azide solution).

Primary and secondary antibodies were diluted in blocking solution and incubated for 2 hours and 1 hour, respectively at room temperature, with 3 intervening washes in PBS. Thy1-CRP⁺ transgenic mice have been genetically engineered to express the cyan fluorescent protein (CFP) reporter gene in retinal ganglion cells. RGC/mm² were quantified in retinas from each experimental group as described in Example 2. The data represent RGD levels in the density of retinal ganglion cells neuronal cells (TuJ-1⁺ cells;) or ganglion cells (Thy1:CFP⁺ cells). After mice experienced 8 weeks of exposure to increased IOP, the density of RGCs in wholemount preparations of mouse retinas was significantly reduced in both neuronal and ganglion cells. RGC loss was rescued by Compound 1 in the retinas from in both cell types, as shown by the higher levels of both RGC markers in MB-injected mice and that had been treated with Compound 1.

### Example 7

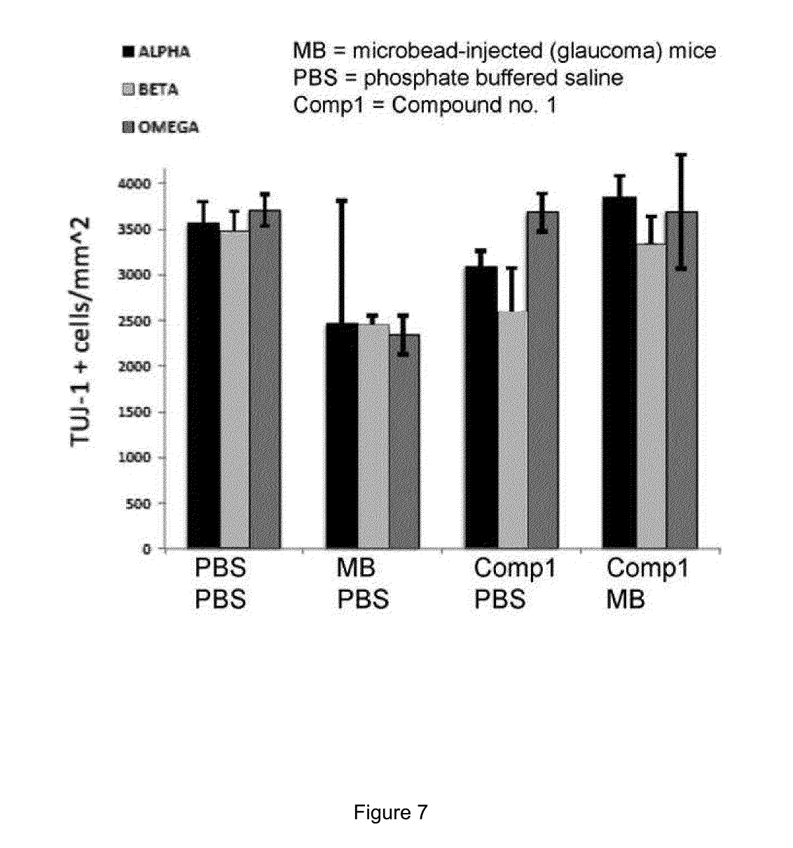

FIG. 7 illustrates the location of affected TUJ-1⁺ cells in relation to the optic nerve in the presence and absence of an exemplary TRPV4 antagonist. MBs or PBS were injected into the anterior chamber of mice eye as described in Example 2. Mice were dosed with Compound 1 or PBS vehicle. Tuj-1 is a marker protein which, in the retina, is selectively expressed in retinal ganglion cells (RGCs). TuJ-1⁺ cells were quantified in the alpha, beta, and omega regions of the eye in each experimental group. There were fewer TuJ-1⁺ cells in eyes from the glaucoma model (mice injected with MB) that were treated with IP injection of vehicle (PBS), relative to the other experimental groups. However, there was no significant difference in TuJ-1⁺ cells between alpha, beta, or omega regions in any experimental group. These data show that the TRPV4 antagonist rescues RGCs across all regions of the retina rather than showing quadrant-specific rescue.

### Example 8

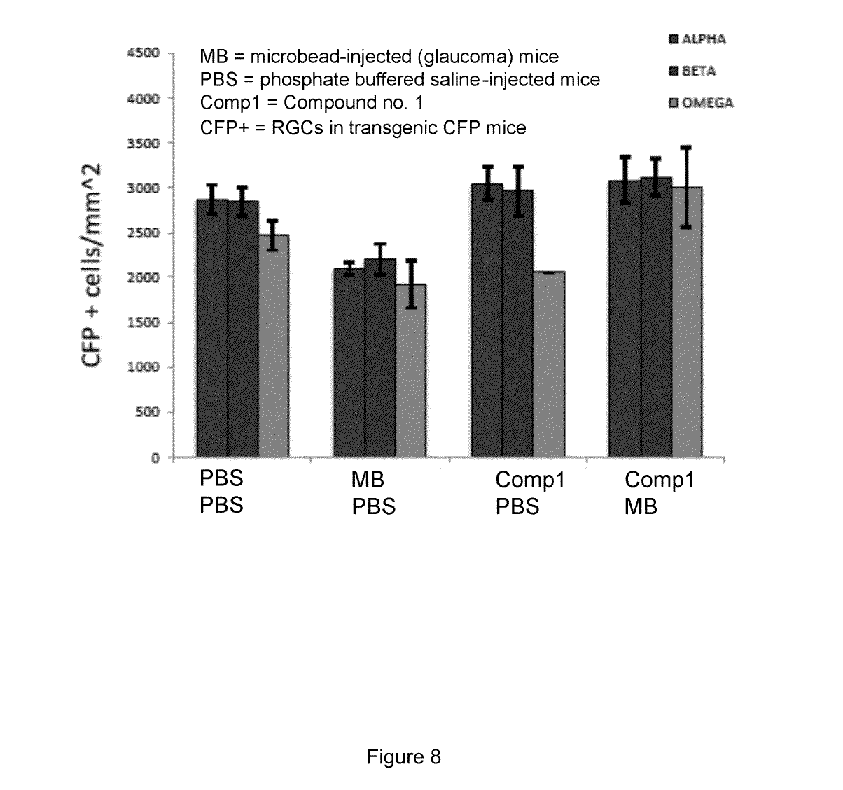

FIG. 8 illustrates the location of RGCs expressing the CFP⁺ reporter transgene in retinas isolated from Thy1-CRP⁺ transgenic mice. The mice were treated as described in Example 7 and the CFP⁺ cells quantified in the alpha, beta, and omega regions within the eye. The omega region showed fewer CFP⁺ cells than alpha or beta in mice that received vehicle injections in the eye and IP compound 1 injections. There was no difference in CFP⁺ cells between alpha, beta, and omega regions in any other treatment group. As in Example 7, these data show that the TRPV4 antagonist rescues RGCs across all regions of the retina rather than showing quadrant-specific rescue.

### Example 9

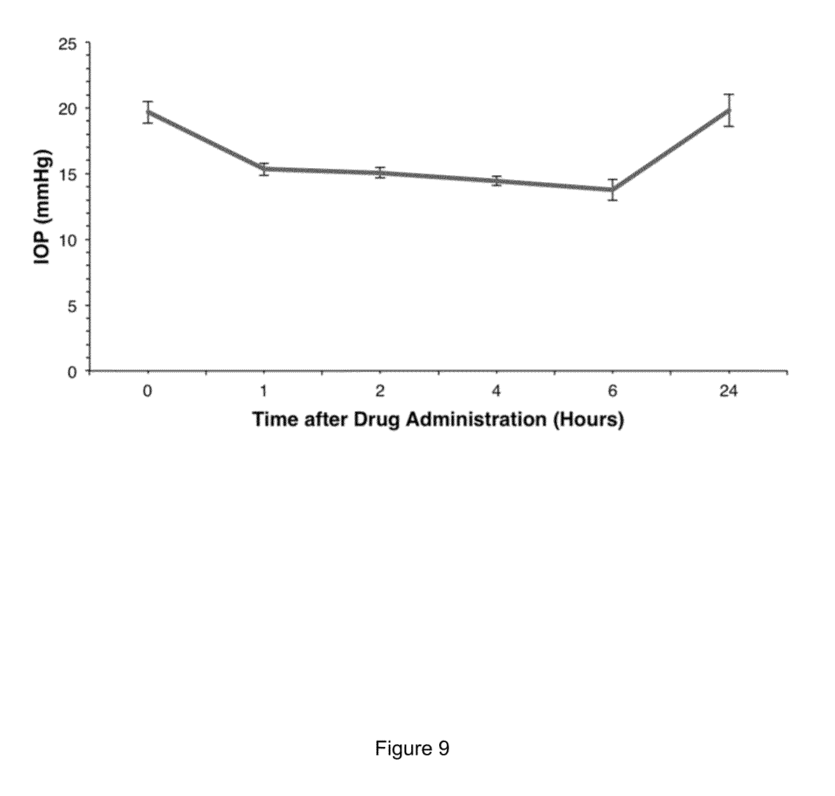

FIG. 9 illustrates the short-term effects of a single dose of Compound 1 (eye drop) in the MB model of glaucoma. IOP was measured as described in Example 3 at the indicated time points after receiving a drop of Compound 1. Compound 1 reduced IOP beginning at 1 hour after dosing and continued through 6 hours after dosing.

### Example 10

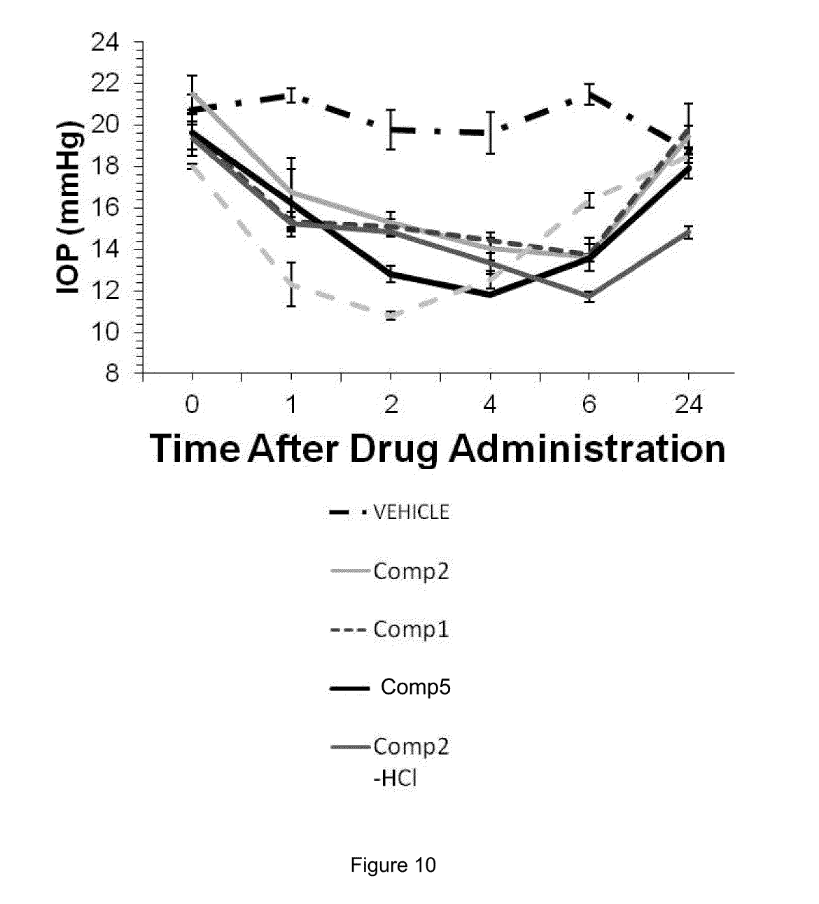

FIG. 10 illustrates a time course of Compounds 1, 2, 5 and Compound 2-HCl as well as a positive control compound, timolol, with regard to inhibition of IOP. The experiment was conducted as described in Example 9. Each of the TRPV4 antagonists reduced IOP with a similar time course. Specifically, Compounds 1, 2, 5 and Compound 2-HCl reduced IOP for at least 6 hours after dosing. This experiment shows that timolol has a left-shifted dose-response curve relative to the exemplary TRPV4 antagonists.

### Example 11

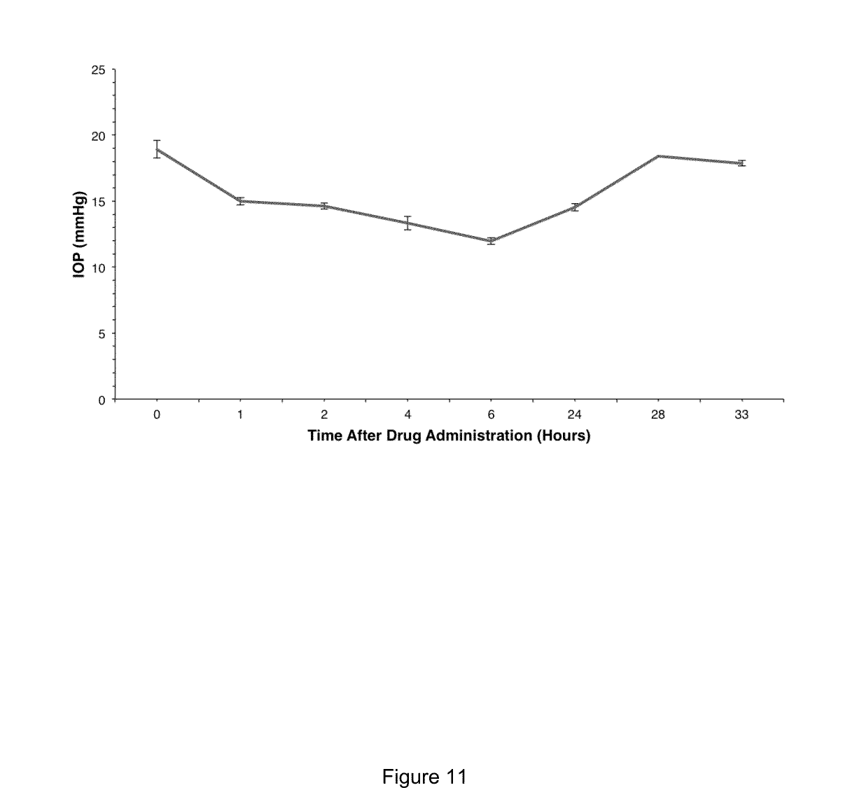

FIG. 11 illustrates a time course of Compound 2-HCl activity. The experiment was conducted as described in Example 9. Compound 2-HCl maximally inhibited IOP at 6 hours after dosing. IOP did not reach pre-dose levels until 33 hours post-dosing. 2

### Example 12

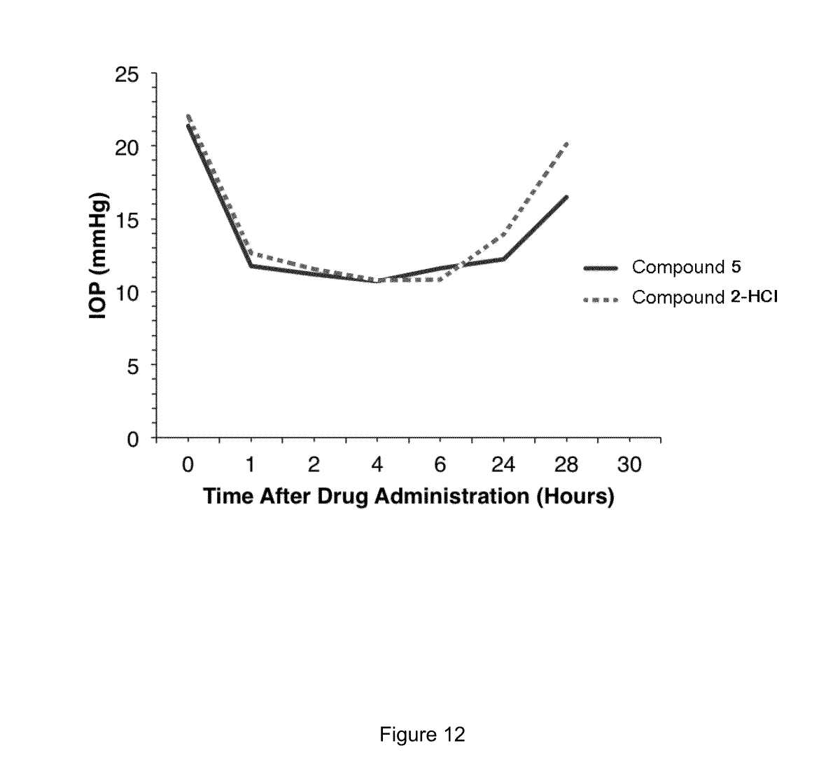

FIG. 12 illustrates a comparison of the time course of Compound 2-HCl and Compound 5. The experiment was conducted as described in Example 9. Both compounds inhibited IOP within 1 hour post treatment. The two compounds showed similar efficacy.

### Example 13

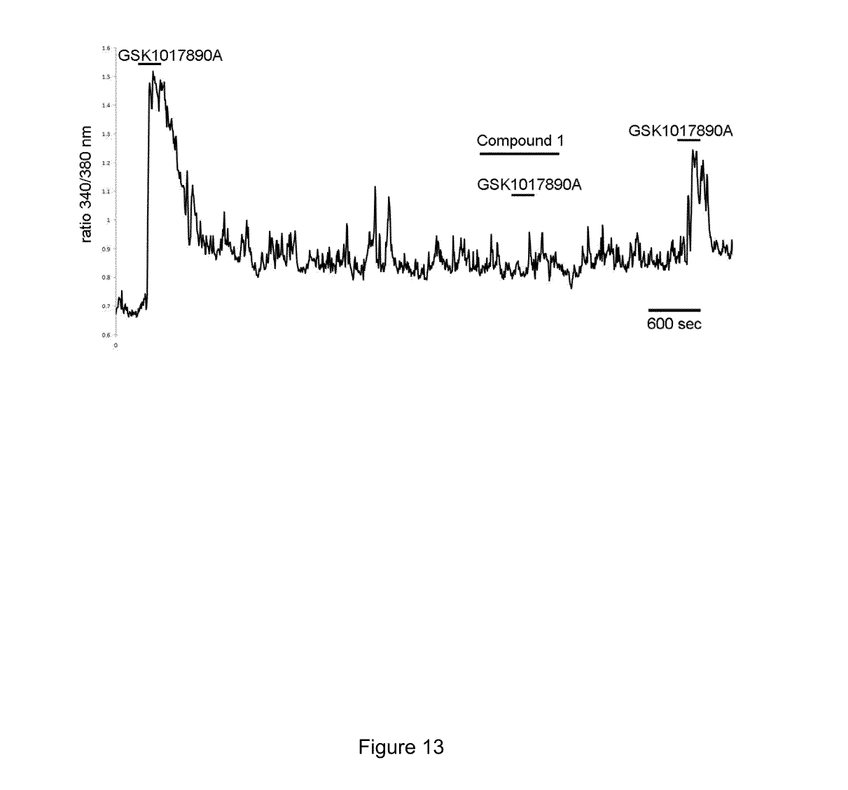

FIG. 13 shows that the TRPV4 agonist GSK evokes sustained increases in intracellular calcium concentration [Ca^{++]}ᵢ in retinal Müller glial cells and this effect is blocked by Compound 1.

### Example 14

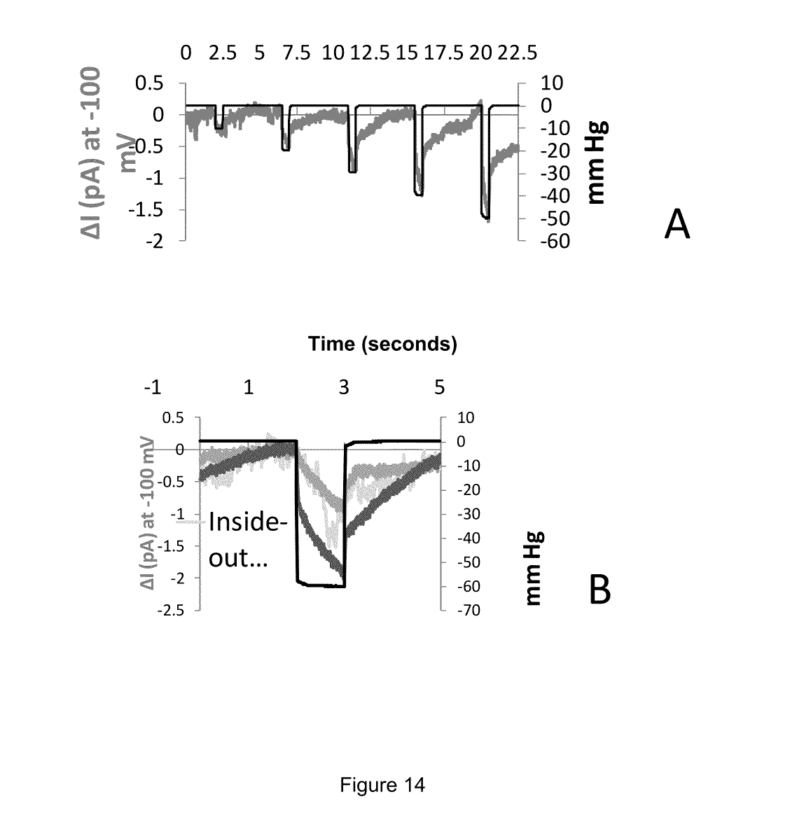

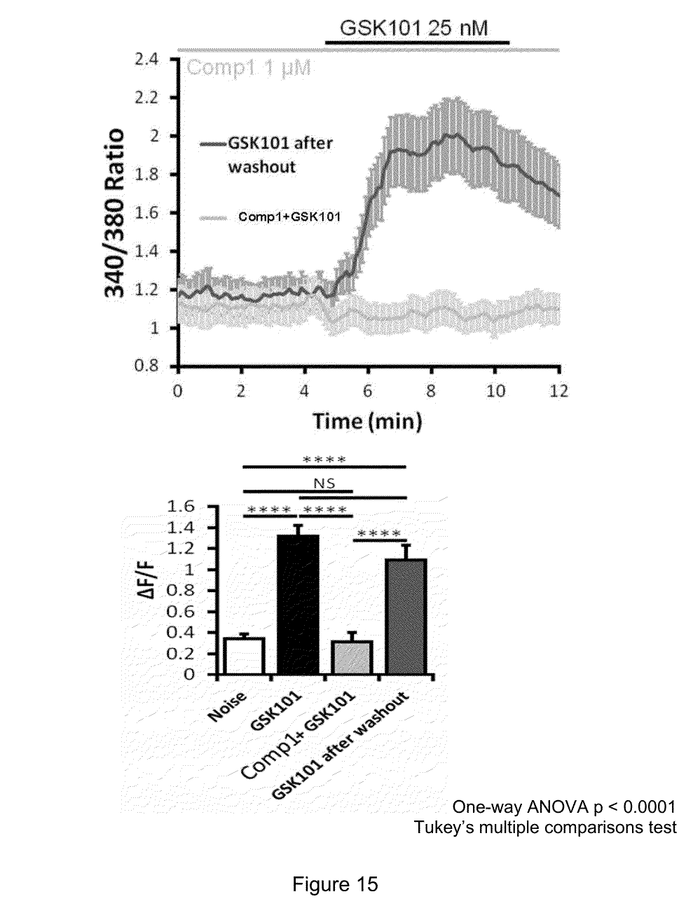

FIG. 14 shows that steps of pressure from 10 mm Hg to 50 mm Hg induce inward currents from a retinal ganglion cell (FIG. 15A). The cell was voltage clamped and stimulated with the High-Speed Pressure Clamp method, as detailed by Besch et al (Pflugers Arch. 2002 October; 445(1):161-6). This method consists of a specialized headstage coupled to a piezo element in the pressure/vacuum control valve. By balancing the access of pressure to the patch pipette, the device generates rapid and reproducible pressure steps of >±200 mm Hg. Channel activity is recorded in the cell-attached mode or the membrane is “zapped” into the outside-out configuration. Proof of principal is provided in FIG. 15A which demonstrates that increased pressure steps elicited increasingly larger inward currents. FIG. 15B shows that the response was reversibly antagonized by compound 1 as illustrated by its ability to block pressure-induced inward currents.

### Example 15

FIG. 15 shows the effect of an exemplary TRPV4 antagonist blocking the effect of a TRPV4 agonist. The TRPV4 agonist GSK evokes sustained increases in intracellular calcium concentration [Ca^{++]}ᵢ in retinal Müller glial cells and this effect is blocked by Compound 1. The effect of Compound 1 on glial calcium levels is reversible, as it can be washed out by 10 minute superfusion with control saline. These effects are highly statistically significant (***=P<0.001).

### Example 16

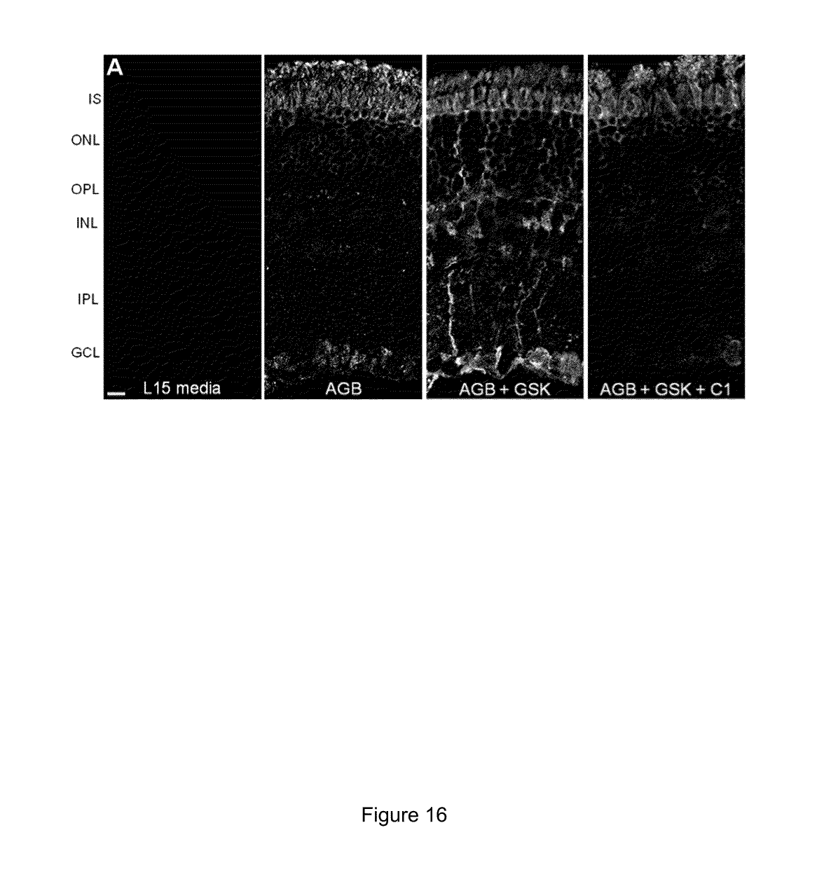

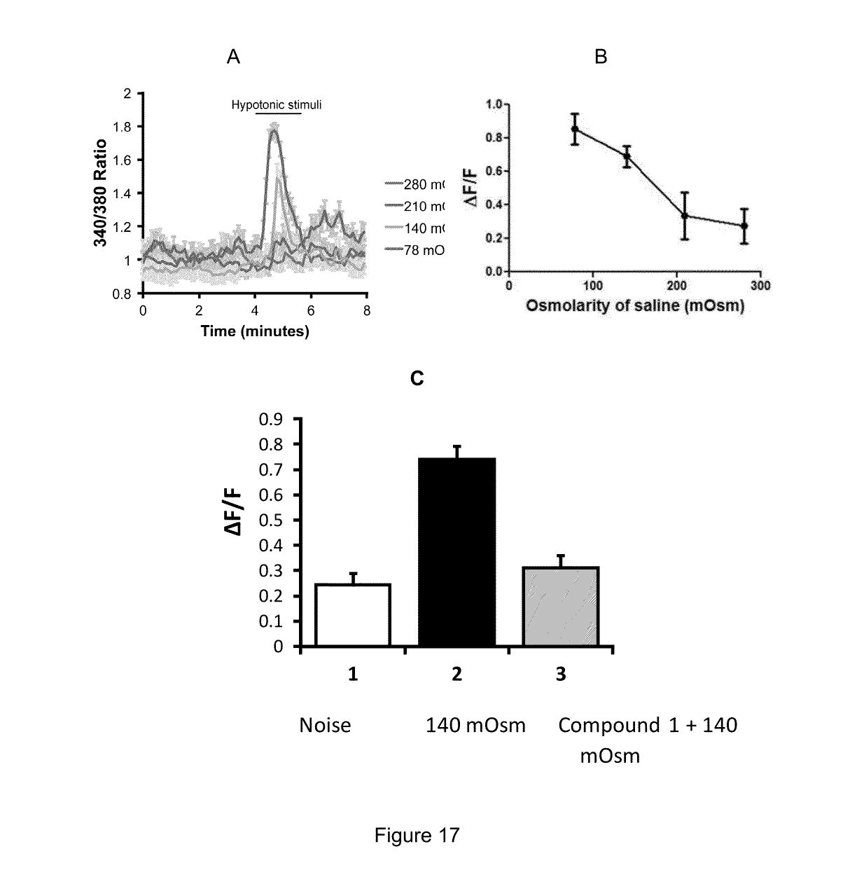

FIG. 16 illustrates the effect of the TRPV4 agonist GSK on cation influx into mouse retinal cells of the mouse retina. The influx was determined using the anti-AGB antibody, as described by Marc RE (J Comp Neurol 407(1): 47-64, 1999). AGB is a membrane permeant cation that can flow through most known cation channels. Using an anti-AGB antibody, it can be determined where the cation flux occurs and, therefore, which cells are activated. As shown in FIG. 17, exposure to the TRPV agonist GSK increased the number of AGB⁺ retinal ganglion cells and Müller glial cells, indicating that these two cell types express TRPV4 channels in the mouse retina. The effect of GSK was strongly antagonized by compound 1 (C1).

### Example 17

FIG. 17 shows that cell swelling is accompanied by a pressure-dependent influx of calcium into retinal ganglion cells (FIGS. 17A-C). Cells were labeled with fluorescent calcium indicator dye Fura-2 and exposed to hypotonic solutions in which extracellular sodium was replaced by the osmotically inert substance mannitol, as described in Ryskamp et al. (J. Neurosci. 2011 May 11; 31(19):7089-101; PMID 21562271). The effect of cell swelling induced by 140 mOsm saline on intracellular calcium levels in ganglion cells was blocked by Compound 1. FIG. 17C shows that compound 1 prevents the pressure-dependent influx of calcium in RGCs.

### Example 18

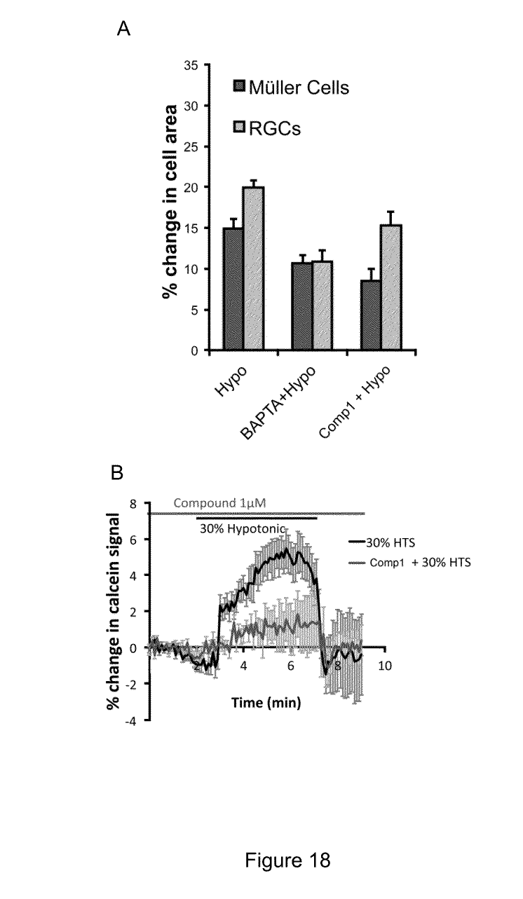

FIG. 18A illustrates the effect of hypotonic stimuli on cell swelling as measured by change in cell area. Hypotonic stimuli were generated by replacing extracellular sodium levels to lower osmolarity from 300 mOsm to 190 mOsm. Cell swelling was significantly reduced by the calcium chelator BAPTA-AM. Compound 1 prevented hypotonic stimuli-mediated cell swelling to the same extent as BAPTA-AM. This experiment shows that cell swelling in retinal neurons (RGCs) and glia (Müller cells) is driven by a TRPV4-mediated mechanism. Furthermore, these data show and that Compound 1 is an effective antagonist of swelling such as might occur in retinal ischemia or diabetic neuropathy. FIG. 18B similarly shows that hypotonic stimuli increase [Ca⁺⁺]ᵢ, which is inhibited by Compound 1.

### Example 19

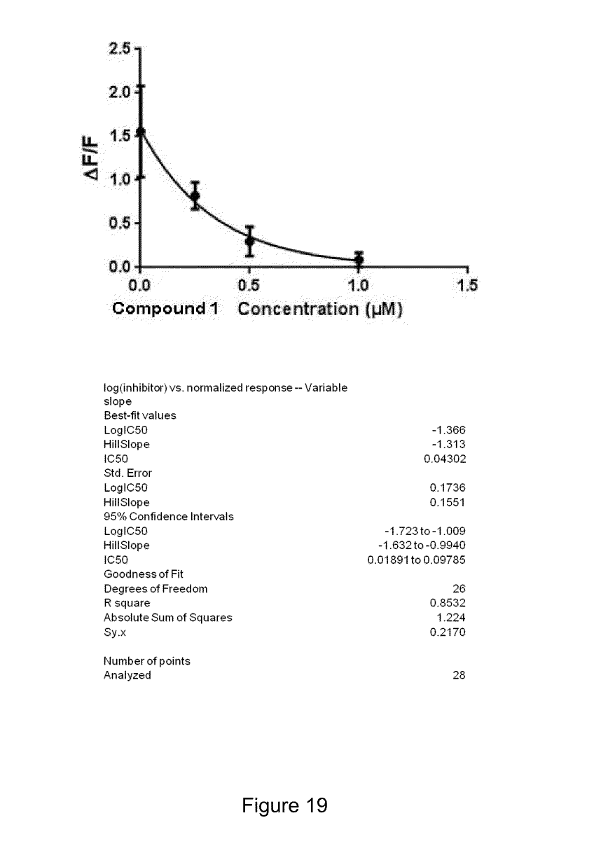

FIG. 19 shows the effectiveness of Compound 1 as a blocker of TRPV4 agonist-induced calcium responses plotted as increased fluorescence (deltaF/F) ratio. Calcium responses in retinal ganglion cells were elicited by 25 nM agonist GSK in the presence of varying concentrations of Compound 1. The half-maximal dose of inhibition was ˜300 nM.

### Example 20

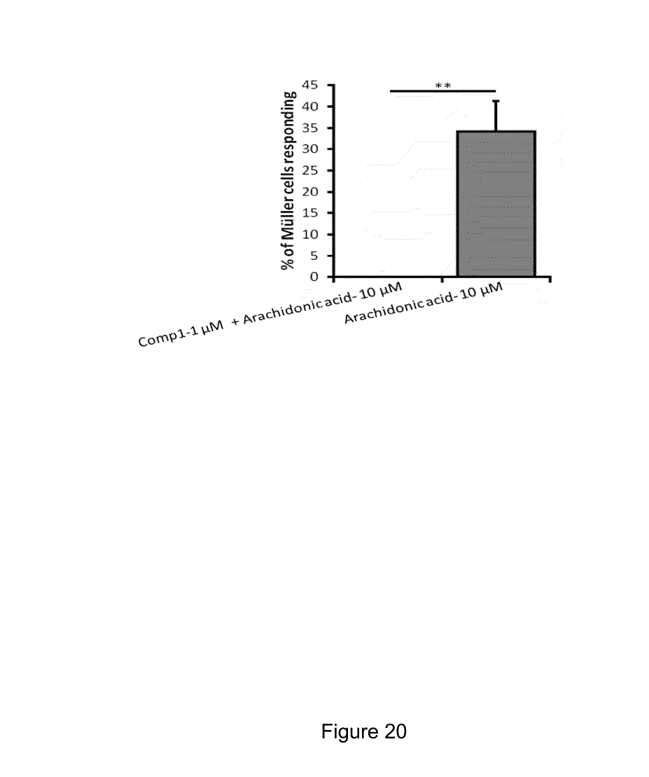

FIG. 20 shows that Müller cells treated with 10 μM arachidonic acid respond with an increase in [Ca2+]ᵢ. This response is blocked by Compound 1 (1 μM).

### Example 21

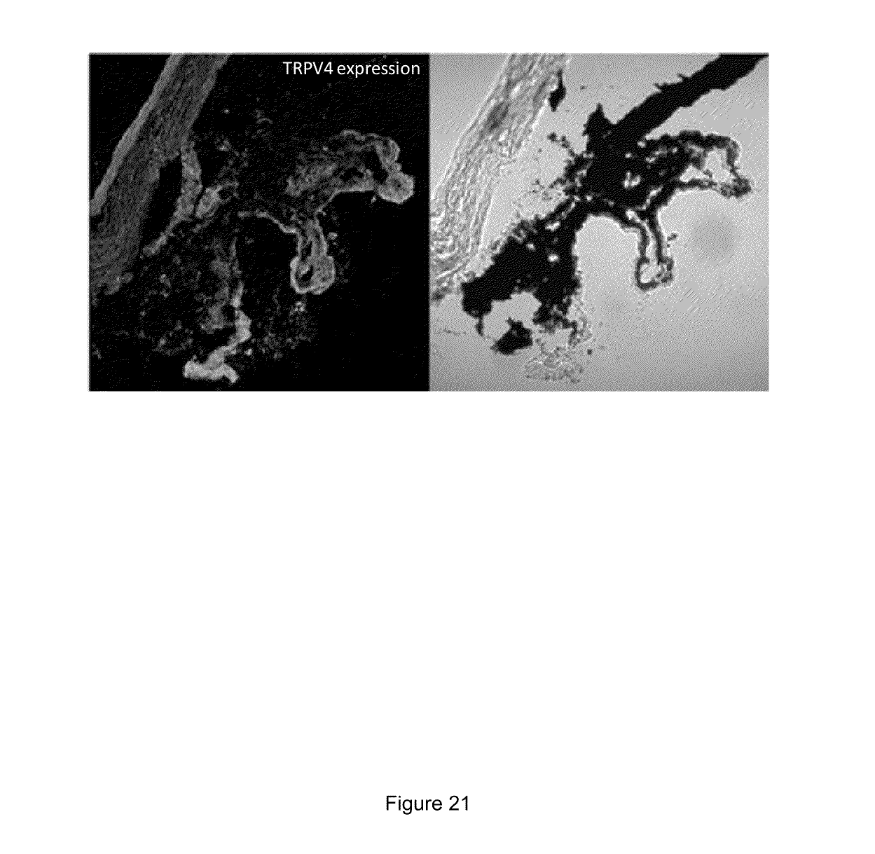

FIG. 21 illustrates that the tissues in the anterior chamber of the eye express the TRPV4 channel. The mouse eye was enucleated, fixed, cryopreserved and sectioned as described in Example 2. The anterior chamber was labeled with a polyclonal rabbit anti-TRPV4 antibody. The antibody stained the ciliary body and trabecular meshwork. This experiment shows that two critical regions, responsible for aqueous humor generation (ciliary body) and primary, conventional, outflow (trabecular meshwork), express TRPV4 channels.

### Example 22

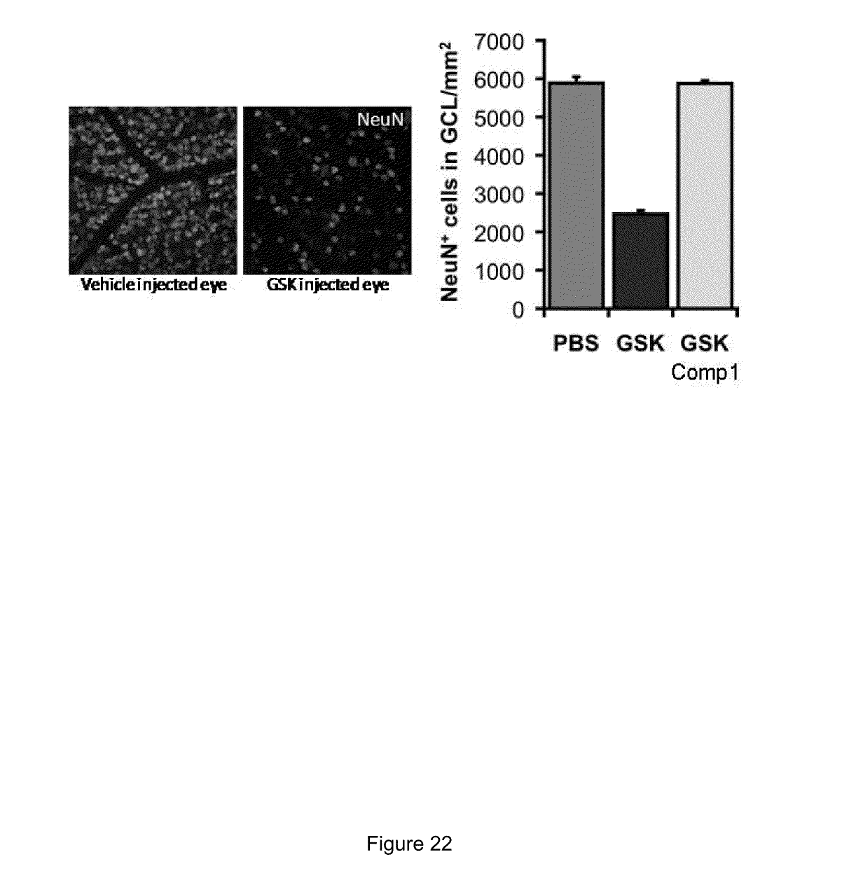

FIG. 22 shows that in vivo intraocular injection of the TRPV4 agonist GSK substantially reduces the number of retinal ganglion cells. In this experiment, GSK (100-300 nM) was injected intraocularly with a Hamilton syringe. Retinas were isolated after 48 hours, fixed and labeled with the RGC marker antibody NeuN. NeuN-immunopositive somata in the ganglion cell layer, shown in the panel on the right, were counted. The bar graph represents quantification of NeuN⁺ cells in the indicated experimental groups. As is evident from the figure, exposure to the TRPV4 agonist induced massive degeneration of RGC. This effect was blocked by Compound 1. This experiment demonstrates that excessive activation of the mechanosensitive channel TRPV4 is sufficient to cause RGC degeneration, an effect that mimics that of excessive pressure on RGCs. Second, this experiment shows the strong neuroprotective action of Compound 1.

### Example 23

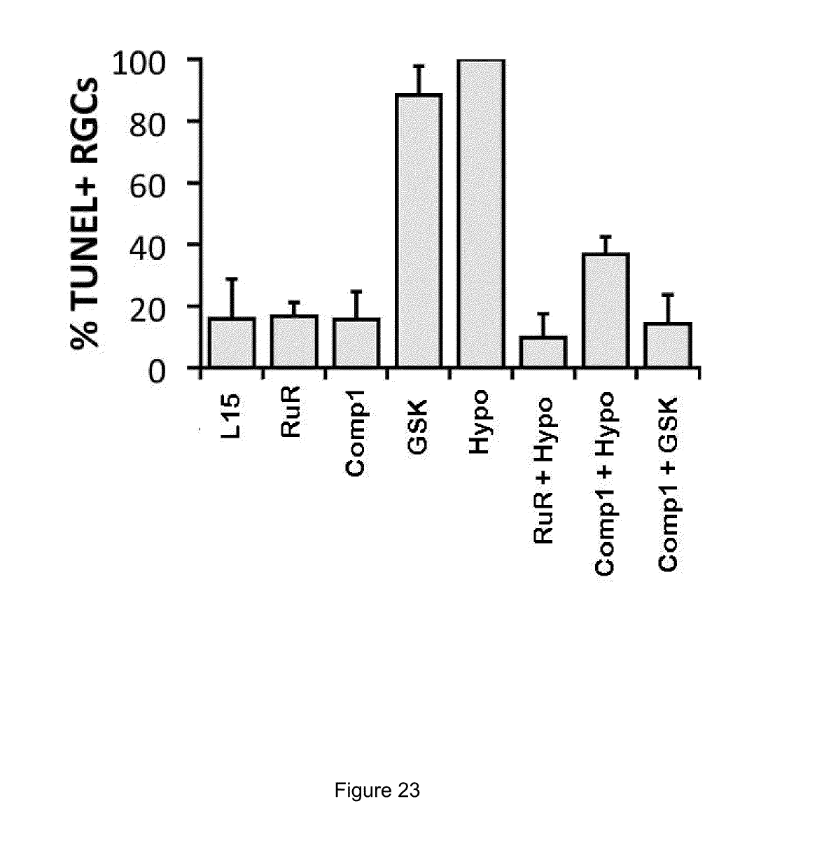

FIG. 23 shows that Compound 1 (1 uM) and the nonselective TRP channel antagonist Ruthenium Red (RuR; 10 μM) block apoptotic cell death induced by the TRPV4 agonist GSK or hypotonic swelling induced by 190 mOsm saline. Terminal deoxynucleotidyl transferase-mediated dUTP-biotin nick end labeling (TUNEL) is a common method for detecting fragmentation that results from apoptotic signaling cascades. Retinas were embedded, cryosectioned, and processed for the TUNEL assay following the protocol by Gavrieli et al. (1992). After 30 min. rehydration in 70% ethanol, 2×5 min. rinses in PBS and 1% Triton-X in 1% citrate buffer (pH 7.3), the slides were subjected to a final rinse in PBS. Slides were then incubated for 30 min. in the reaction buffer (30 mM Trizma-HCl, 140 mM Na⁺ cacodylate, 1.0 mM CaCl₂, 0.2% Triton X-100, pH 7.2). Positive controls were treated with DNAse I (Roche Diagnostics; Branford, Conn.; 10 U/ml) in the reaction buffer. Next, slides were treated for 1 hour with 0.03 units/pi terminal transferase and 4 μM biotin-16-dUTP (Roche Diagnostics). The reaction was terminated in 30 mM Na+ citrate, 300 mM NaCl and 0.2% Triton X-100 in PBS (5 min.) and rinsed in PBS (2×5 min), exposed to 1% bovine serum albumin in PBS for 20 min and rinsed. Apoptotic cells were visualized with a confocal microscope (Zeiss LSM510) and categorized by size. Both GSK (100 nM) and hypotonic stimulation (190 mOsm) induced death of retinal ganglion cells manifested as increased TUNEL staining. This was blocked by RuR and by the TRPV4 antagonist Compound 1.

### Example 24

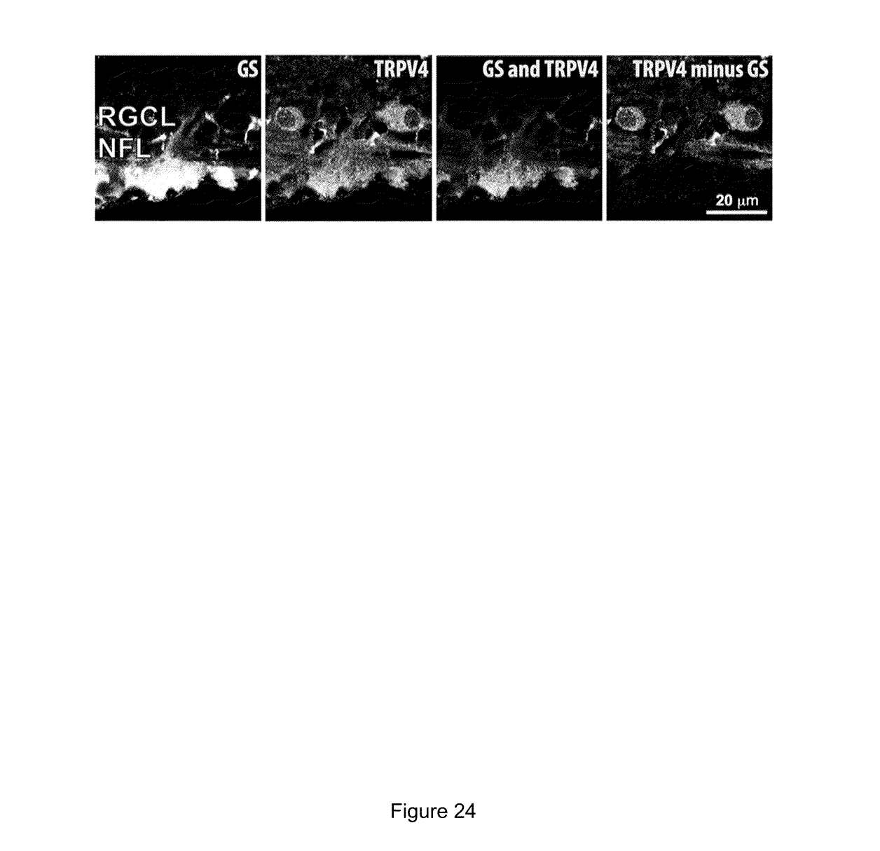

FIG. 24 shows that the human retina shows similar TRPV4 expression compared to the mouse retina. Retina obtained from human donor was immunostained with a polyclonal anti-TRPV4 antibody and a monoclonal mouse glutamine synthetase antibody (a marker for retinal glial cells). This was followed by labeling with anti-rabbit ALEXA 488 nm-conjugated and anti-mouse 594 nm-conjugated secondary antibodies, as detailed in Ryskamp et al. (J. Neurosci. 2011 May 11; 31(19):7089-101). FIG. 24 shows that retinal ganglion cells and Müller glial endfeet processes strongly express TRPV4, as manifested by labeling with the TRPV4-specific antibody.

One embodiment disclosed herein includes administering at least one of the compounds disclosed herein to a subject determined to be in need of treatment for glaucoma. In an embodiment, a method of treating an ocular disease comprises administering a therapeutically effective amount of a compound of Formula 1. In certain embodiments, the compound is administered topically, periocularly or intraocularly. In some embodiments, the compound is administered via an intraocular injection.

In further embodiments, the compounds and pharmaceutical compositions disclosed herein may be coadministered with another pharmaceutically active compound. For example, the compounds may be coadministered with antibiotics, anti-inflammatory agents, anesthetics, steroids, carbonic anhydrase inhibitors, beta-adrenergic receptor antagonists, vasodilators and/or anti-viral agents, or any combination or mixtures of these, whether administered separately or in a single pharmaceutical composition. In an embodiment, compositions may comprise a compound of Formula I and a compound chosen from at least one timolol, dexamethasone, prednisone, brimonidine, dorzolamide, travoprost, bimatoprost, pilocarpine and lantanoprost.

In certain embodiments, a combination therapy may involve treating the individual in need of treatment with a compound of Formula I in combination with a steroid, carbonic anhydrase inhibitor, beta-adrenergic receptor antagonist, or vasodilator, or any combination or mixtures of these. A method of treating an ocular disease may comprise administering a therapeutically effective amount of a compound of claim 1, and further comprise administering a therapeutically effective amount of at least one of an antibiotic, an anti-inflammatory agent, an anesthetic, a steroid, a carbonic anhydrase inhibitor, a beta-adrenergic receptor antagonist, a vasodilator and an anti-viral agent.

The treatment regime used in the combination therapy can involve administration of a composition comprising the combination of active ingredients, or the concomitant administration of separate compositions, each comprising at least one active ingredient. Furthermore, the administration of the active ingredients can be performed at different times and/or via different routes. For example, a composition comprising at least one active ingredient can be administered in the morning, and a composition comprising at least one different active ingredient can be administered in the evening. Another embodiment involves the administration of a composition having at least one active ingredient topically while the second composition is administered intraocularly.

In certain embodiments, a method of treating an ocular disease comprises administering a therapeutically effective amount of a compound of claim 1 once-daily, or qd. In some embodiments, the administering may be twice a day (bid), three times a day (tid) or four times a day (qid). The administering may be achieved via any of the routes described herein, including topical, periocular or intraocular administration. For example, a method of treating an ocular disease may comprise administering a therapeutically effective amount of a compound of claim 1 once-daily by injection, such as via a subconjunctival, retrobulbar, juxtascleral or intravitreal injection. In some embodiments, the administering is achieved via an ophthalmic device, such as an implant, a topically applied composition, such as an eyedrop, or a gel-forming solution, any of which may occur once-daily (qd), twice a day (bid), three times a day (tid) or four times a day (qid).

The invention further provides a strategy for treating and/or preventing abnormal IOP, which is particularly relevant for patients with “low-tension” glaucoma. According to this aspect of the invention, an individual who may be predisposed to abnormal IOP is administered a compound of Formula I. In an embodiment, the preventative therapy involves treating the individual in need of treatment with a compound of Formula I in combination with antibiotics, anti-inflammatory agents, anesthetics, steroids, carbonic anhydrase inhibitors, beta-adrenergic receptor antagonists, vasodilators and/or anti-viral agents, or any combination or mixtures of these, as described above. For example, a preventative therapy can involve treating the individual in need of treatment with a compound of Formula I in combination with a compound chosen from at least one of timolol, dexamethasone, prednisone, brimonidine, dorzolamide, travoprost and lantanoprost.

The skilled artisan readily recognizes that the invention includes the use of compounds of Formula I, pharmaceutically acceptable salts, metabolites and prodrugs thereof for treating ocular disease. In accordance with the various treatment methods of the disclosure, the compound of Formula I may be delivered to a subject in a manner consistent with conventional methodologies associated with management of the disorder for which treatment or prevention is sought.

Typical subjects intended for treatment with the compounds, compositions and methods of the present disclosure include humans, as well as non-human primates and other animals such as companion animals including birds, marsupials, livestock animals, animals used in models of ocular diseases, or animals used in pharmaceutical testing, such as pharmacokinetics and toxicological testing, including mice, rats, rabbits, and guinea pigs. To identify subjects for prophylaxis or treatment according to the methods of the disclosure, accepted screening methods are employed to determine the status of an existing or likely ocular disease or condition in a subject. These screening methods include, for example, measurement of the subjects' IOP. These and other routine methods allow a clinician to select subjects in need of therapy using the methods and pharmaceutical compositions of the disclosure.

Genetic screening may be used to identify subjects in need of therapy using the methods and pharmaceutical compositions of the disclosure. For example, subjects with a family history of glaucoma may be screened for markers indicating an increased likelihood of having glaucoma. Screening for mutations in the TRPV4 would also identify subjects for prophylaxis or treatment according to the methods of the disclosure.

In an embodiment, a method of treating an ocular disease comprises administering a therapeutically effective amount of a compound of Formula 1, wherein the ocular disease is selected from at least one of retinopathies including non-proliferative and proliferative diabetic retinopathy and retinopathy of prematurity, glaucoma, macular degeneration, age-related macular degeneration (wet and dry), retinitis pigmentosa, Stargardt disease, macular edema, uveitis, and retinal infections including those with cytomegalovirus. In certain embodiments, the ocular disease is at least one of diabetic retinopathy or glaucoma.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated. The scope of the invention is therefore defined by the following claims.

1. A compound of Formula I: 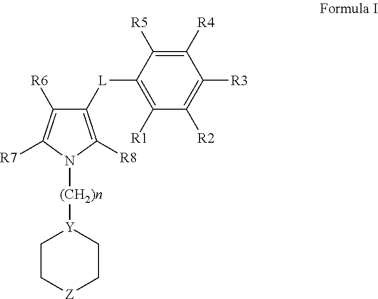
wherein L is C(═O)NR10, SO₂NR10, a C1-C6 alkylene, or a bond;
Y is N or CR10;
Z is O, NR10, S, SO₂ or C(R10)₂;
n is 0, 1, 2, 3, 4, 5, or 6;
R1, R2, R3, R4, and R5 are independently selected from at least one of hydro, alkyl, haloalkyl, hydroxy, alkoxy, haloalkoxy, cyano, carboxyl, carboxyalkyl or amido;
R6 and R7 are independently selected from at least one of hydro, alkyl, haloalkyl, heterocyclic or aryl, or R6 and R7 are connected to via a cyclic ring system;
R8 is hydro, alkyl, haloalkyl, heterocyclic or aryl; and
R10 is hydro, alkyl, haloalkyl, carboxyalkyl, carboxyl, alkyl methylene carbonate, methylene carbamyl, thiophenyl or —S-carboxyalkyl;
or pharmaceutically acceptable salts thereof.

2. The compounds of claim 1, wherein
L is C(═O)NH or SO₂NH;
Y is N;
Z is O, N(C1-C6 alkyl), SO₂ or CH₂;
n is 3;
R1, R3, R4 and R5 are hydro;
R2 is haloalkyl, haloalkoxy, carboxyl, cyano or amido;
R6 is hydro, R7 is aryl, or
R6 and R7 are connected via a cyclic ring system; and
R8 is alkyl.

3. The compounds of claim 1, wherein
L is C(═O)NH;
Y is N;
Z is O;
n is 3;
R1, R3, R4 and R5 are hydro;
R2 is OCF₃, CF₃, CONR₂ or COOR, where R is hydro or alkyl;
R6 is hydro, R7 is phenyl, or
R6 and R7 are connected via a cyclic ring system; and
R8 is methyl.

4. The compounds of claim 1, wherein
L is C(═O)NR10;
Y is N;
Z is O;
n is 3;
R1, R3, R4 and R5 are hydro;
R2 is OCF₃, CF₃, CONR₂ or COOR, where R is hydro or alkyl;
R6 is hydro, R7 is phenyl, or
R6 and R7 are connected via a cyclic ring system;
R8 is methyl; and
R10 is carboxyalkyl, carboxyl, alkyl methylene carbonate, methylene carbamyl, thiophenyl or —S-carboxyalkyl.

5. The compounds of claim 1, wherein the compounds have the structure of Formula II: 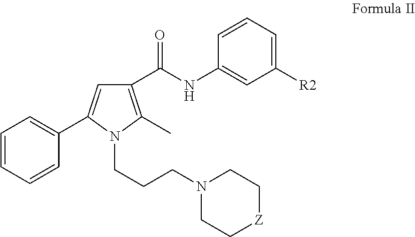
wherein Z is O, NR10, S, SO₂ or C(R10)₂;
R2 is selected from at least one of hydro, alkyl, haloalkyl, hydroxy, alkoxy, haloalkoxy, cyano, carboxyl, carboxyalkyl or amido; and
R10 is hydro, alkyl, or haloalkyl.

6. The compounds of claim 5, wherein
Z is O, N(C1-C6 alkyl), SO₂ or CH₂; and
R2 is OCF₃, CF₃ or COOR, wherein R is hydro or alkyl.

7. The compounds of claim 5, wherein
Z is O; and
R2 is OCF₃, CF₃, or COOR, wherein R is isopropyl.

8. The compounds of claim 1, wherein the compounds have the structure of Formula III: 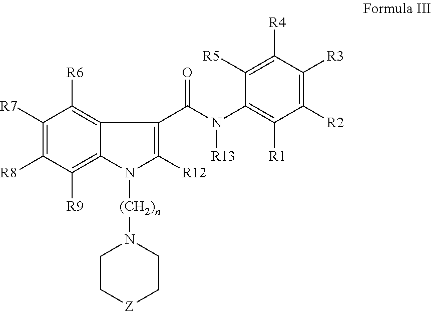
wherein Z is O, NR10, S, SO₂ or C(R10)₂;
n is between 0 and 6;
R1—R9 and R12 are selected from at least one of hydro, alkyl, haloalkyl, hydroxy, alkoxy, haloalkoxy, cyano, carboxyl, or amido;
R10 is hydro, alkyl, or haloalkyl; and
R13 is hydro, alkyl, haloalkyl, carboxyalkyl, carboxyl, alkyl methylene carbonate, methylene carbamyl, thiophenyl or —S-carboxyalkyl.

9. The compounds of claim 8, wherein
Z is O, N(C1-C6 alkyl), SO₂ or CH₂;
n is 3;
R2 is OCF₃, CF₃ or COOR, wherein R is hydro or alkyl;
R9 is halo or alkyl;
R12 is alkyl; and
R13 is hydro.

10. The compounds of claim 8, wherein
Z is O;
n is 3;
R2 is OCF₃, CF₃, or COOR, wherein R is isopropyl, ethyl, butyl, isobutyl, n-propyl or methyl;
R9 is chloro or tert-butyl;
R12 is methyl; and
R13 is hydro.

11. The compounds of claim 8, wherein
Z is O;
n is 3;
R2 is OCF₃, CF₃, or COOR, wherein R is isopropyl, ethyl, butyl, isobutyl, n-propyl or methyl;
R9 is chloro or tert-butyl;
R12 is methyl; and
R13 is carboxyalkyl, carboxyl, alkyl methylene carbonate, methylene carbamyl, thiophenyl or —S-carboxyalkyl.

12. The compound of claim 1, wherein the compound is selected from one of:
N-(3-(trifluoromethyl)phenyl)-2-methyl-1-(3-morpholinopropyl)-5-phenyl-1H-pyrrole-3-carboxamide;
iso-propyl 3-(2-methyl-1-(3-morpholinopropyl)-5-phenyl-1H-pyrrole-3-carboxamido)benzoate;
2-methyl-1-(3-morpholinopropyl)-5-phenyl-N-(3-(trifluoromethoxy)phenyl)-1H-pyrrole-3-carboxamide; and
N-(3-(trifluoromethyl)phenyl)-2-methyl-1-(3-morpholinopropyl)-1H-indole-3-carboxamide.

13. A method of preparing substituted N-(3-morpholinopropyl)-5-phenyl-1H-pyrrole-3-carboxamides according to claim 4, comprising reacting substituted 2-acetyl-4-oxo-4-phenyl butanoates with 3-morpholinopropan-1-amine, and reacting substituted N-(3-morpholinopropyl)-5-phenyl-1H-pyrrole-3-carboxylates with substituted anilines via an acid chloride.

14. A pharmaceutical composition comprising a compound of claim 1 and at least one excipient.

15. A method of treating an ocular disease in a subject, the method comprising administering a therapeutically effective amount of a compound of claim 1.

16. The method of claim 15, wherein the subject is mammalian, avian or marsupial.

17. The method of claim 16, wherein the subject is primate or human.

18. The method of claim 15, wherein the compound is administered topically, periocularly or intraocularly.

19. The method of claim 15, wherein the compound is administered via an intraocular injection.

20. The method of claim 15, wherein the ocular disease is selected from at least one of retinopathies including non-proliferative and proliferative diabetic retinopathy and retinopathy of prematurity, glaucoma, macular degeneration, age-related macular degeneration (wet and dry), retinitis pigmentosa, Stargardt disease, macular edema, uveitis, and retinal infections including those with cytomegalovirus.

21. The method of claim 15, wherein the ocular disease is at least one of diabetic retinopathy or glaucoma.

22. The method of claim 15, further comprising administering a therapeutically effective amount of at least one of an antibiotic, an anti-inflammatory agent, an anesthetic, a steroid, a carbonic anhydrase inhibitor, a beta-adrenergic receptor antagonist, a vasodilator and an anti-viral agent.

23. The method of claim 15, further comprising administering a therapeutically effective amount of at least one of the following: timolol, dexamethasone, prednisone, brimonidine, dorzolamide, travoprost, bimatoprost, pilocarpine and lantanoprost.
